(19) United States
(12) Patent Application Publication
(10) Pub. No.: US 2025/0262228 A1
(43) Pub. Date: Aug. 21, 2025

(54) TREATMENT OF SPASTICITY AND PAIN IN RENALLY-IMPAIRED PATIENTS

(71) Applicant: Jazz Pharmaceuticals Research UK Limited., Sittingbourne, Kent (GB)

(72) Inventors: Cuiping CHEN, Palo Alto, CA (US); Sigal GAL, Chalfont, PA (US); Alessandro SCHIPANI, Sawston, Cambridgeshire (GB); Franck SKOBIERANDA, Palo Alto, CA (US)

(21) Appl. No.: 18/859,454

(22) PCT Filed: Apr. 26, 2023
(86) PCT No.: PCT/EP2023/061030

§ 371 Date: Oct. 23, 2024

Related U.S. Application Data
(60) Provisional application No. 63/335,370, filed on Apr. 27, 2022.

(51) Int. Cl. A61K 31/00; A61K 9/00; A61K 9/12; A61P 13/12

(52) U.S. Cl. CPC A61K 31/658; A61K 9/006; A61K 9/12; A61P 13/12

(57) ABSTRACT

The present disclosure provides methods of treating multiple sclerosis associated spasticity or neurological pain in a patient with renal impairment by administering cannabinoids oromucosal spray.

### CROSS REFERENCE TO RELATED APPLICATION

The present Application claims priority to U.S. Provisional Application No. 63/335,370, filed Apr. 27, 2022, of which is hereby incorporated by reference in its entirety for all purposes.

### BACKGROUND OF THE INVENTION

Multiple sclerosis (MS) is a chronic disabling disease that affects the brain and spinal cord. The signs and symptoms of MS vary greatly and depend on the extent of nerve damage and the location or types of nerves involved. The exact cause of MS is unknown, and no cure has been found.

To date, a number of FDA-approved MS medications are available to alleviate and manage the symptoms of MS, such as spasticity and pain. However, currently available medications are generally poorly tolerated and have been associated with side effects such as flu-like symptoms, changes in heart rate, chest pain, infection, nausea, and hair loss. Side effects may be exacerbated by a patient's physiology.

Of note, recent studies have shown that impaired renal function or renal failure is prevalent in MS patients undergoing MS therapeutic treatments, as often represented by lowering of the glomerular filtration rate (GFR). To make the matter worse, many of the MS drugs have the potential adverse effect of infections that requires prolonged antibiotic treatment—and antibiotics are known as one of the primary causes of drug-associated nephropathy—which may further deteriorate the kidney function. Thus, there exists a need for safe and effective methods for treating patients with renal impairment.

### SUMMARY OF THE INVENTION

The present disclosure provides safe and effective methods of treating or managing multiple sclerosis (MS) symptoms and pain associated with cancer in patients with renal impairment. More specifically, as described herein, Applicant has discovered a dosing regimen that allows for safely, and effectively treating MS spasticity, spinal cord injury spasticity, MS neuropathic pain, and pain associated with advanced cancer in a patient with mild, moderate and severe renal impairment.

In one aspect, the present disclosure provides a method of treating or managing spasticity in a patient with multiple sclerosis, wherein the patient has moderate or severe renal impairment. In embodiments, the patient is orally administered 1 to 6 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5. In embodiments, the patient is orally administered a maximum of about 6 sprays per day of an oromucosal spray comprising THC and cannabidiol CBD.

In one aspect, the present disclosure provides a method of treating or managing neuropathic pain in a patient with multiple sclerosis, wherein the patient has moderate or severe renal impairment. In embodiments, the patient is orally administering 1 to 6 sprays per day of an oromucosal spray comprising THC and CBD, wherein the THC and CBD are present at a weight ratio ranging from 0.5:1.5 to 1.5:0.5. In embodiments, the patient is orally administered a maximum of about 6 sprays per day of an oromucosal spray THC and CBD.

In one aspect, the present disclosure provides a method of treating or managing pain in a patient with advanced cancer, wherein the patient has moderate or severe renal impairment. In embodiments, the patient is orally administered from about 1 to about 6 sprays per day of an oromucosal spray comprising THC and CBD, wherein the THC and CBD are present at a weight ratio ranging from 0.5:1.5 to 1.5:0.5. In embodiments, the patient is orally administered a maximum of about 6 sprays per day of an oromucosal spray comprising THC and CBD.

In embodiments, the patient has severe renal impairment. In such embodiments, the patient with severe renal impairment is administered 1 spray per day of the oromucosal spray. In embodiments, the patient with severe renal impairment is administered 2 sprays per day of the oromucosal spray. In embodiments, the patient with severe renal impairment is administered 3 sprays per day of the oromucosal spray. In embodiments, the patient with severe renal impairment is administered 4 sprays per day of the oromucosal spray. In embodiments, the patient with severe renal impairment is administered 5 sprays per day of the oromucosal spray. In embodiments, the patient with severe renal impairment is administered 6 sprays per day of the oromucosal spray. In embodiments, the patient has moderate renal impairment. In such embodiments, the patient with moderate renal impairment is administered 1 spray per day of the oromucosal spray. In embodiments, the patient with moderate renal impairment is administered 2 sprays per day of the oromucosal spray. In embodiments, the patient with moderate renal impairment is administered 3 sprays per day of the oromucosal spray. In embodiments, the patient with moderate renal impairment is administered 4 sprays per day of the oromucosal spray. In embodiments, the patient with moderate renal impairment is administered 5 sprays per day of the oromucosal spray. In embodiments, the patient with moderate renal impairment is administered 6 sprays per day of the oromucosal spray. In some embodiments, the patient with severe renal impairment has end stage renal disease (ESRD). In some embodiments, the patient with ESRD does not receive dialysis.

In one aspect, the present disclosure provides a method of treating spasticity in a patient with multiple sclerosis, wherein the patient has mild renal impairment. In embodiments, the patient with mild renal impairment is administered about 1 to about 12 sprays per day of an oromucosal spray comprising THC and CBD, wherein the THC and CBD are present at a weight ratio ranging from 0.5:1.5 to 1.5:0.5. In embodiments, the patient is orally administered a maximum of about 12 sprays per day of an oromucosal spray comprising THC and CBD. In one aspect, the present disclosure provides a method of treating neuropathic pain in a patient with multiple sclerosis, wherein the patient has mild renal impairment. In embodiments, the patient with mild renal impairment is administered about 1 to about 12 sprays per day of an oromucosal spray comprising THC and CBD, wherein the THC and CBD are present at a weight ratio ranging from 0.5:1.5 to 1.5:0.5. In embodiments, the patient is orally administered a maximum of about 12 sprays per day of an oromucosal spray comprising THC and CBD.

In one aspect, the present disclosure provides a method of treating pain in a patient with advanced cancer, wherein the patient has mild renal impairment. In embodiments, the patient with mild renal impairment is administered about 1 to about 12 sprays per day of an oromucosal spray comprising THC and cannabidiol CBD, wherein the THC and CBD are present at a weight ratio ranging from 0.5:1.5 to 1.5:0.5. In embodiments, the patient is orally administered a maximum of about 12 sprays per day of an oromucosal spray comprising THC and CBD.

In embodiments, the patient with mild renal impairment is administered 1 spray per day of the oromucosal spray. In embodiments, the patient with mild renal impairment is administered 2 sprays per day of the oromucosal spray. In embodiments, the patient with mild renal impairment is administered 3 sprays per day of the oromucosal spray. In embodiments, the patient with mild renal impairment is administered 4 sprays per day of the oromucosal spray. In embodiments, the patient with mild renal impairment is administered 5 sprays per day of the oromucosal spray. In embodiments, the patient with mild renal impairment is administered 6 sprays per day of the oromucosal spray. In embodiments, the patient with mild renal impairment is administered 7 sprays per day of the oromucosal spray. In embodiments, the patient with mild renal impairment is administered 8 sprays per day of the oromucosal spray. In embodiments, the patient with mild renal impairment is administered 9 sprays per day of the oromucosal spray. In embodiments, the patient with mild renal impairment is administered 10 sprays per day of the oromucosal spray. In embodiments, the patient with mild renal impairment is administered 11 sprays per day of the oromucosal spray. In embodiments, the patient with mild renal impairment is administered 12 sprays per day of the oromucosal spray.

In embodiments, the oromucosal spray releases or delivers about 50 μL to 200 μL per spray. In embodiments, the oromucosal spray releases or delivers about 100 μL per spray. In embodiments, the oromucosal spray releases or delivers about 2 to about 3 mg of THC and about 2 to about 3 mg of CBD per spray. In embodiments, oromucosal spray releases or delivers about 2.25 to about 2.75 mg of THC and about 2.25 to about 2.75 mg of CBD per spray. In embodiments, the patient is administered about 2 to about 3 mg of THC and about 2 to about 3 mg of CBD per spray. In embodiments, the patient is administered about 2.25 to about 2.75 mg of THC and about 2.25 to about 2.75 mg of CBD per spray.

In embodiments, the oromucosal spray comprises from 20-30 mg/mL THC and from 20-30 mg/mL CBD. In embodiments, the oromucosal spray comprises from 22.5-27.5 mg/mL THC and from 22.5-27.5 mg/mL CBD. In embodiments, the oromucosal comprises about 27 mg/mL THC and about 25 mg/mL CBD. In embodiments, the THC and CBD are present in a ratio of from 0.9:1.1 to 1.1:0.9. In embodiments, the oromucosal spray comprises ethanol. In embodiments, the oromucosal spray comprises glycol. In embodiments, the oromucosal spray further comprises peppermint oil.

In another aspect, the present disclosure further provides a method of managing treatment of spasticity in a renally impaired patient with multiple sclerosis, neuropathic pain in a renally impaired patient with multiple sclerosis, or pain in a renally impaired patient with advanced cancer, in which the method comprises the steps of (a) determining the patient's renal impairment status; (i) if the patient has moderate or severe renal impairment, administering about 1 to about 6 sprays per day of an oromucosal comprising THC and CBD; or (ii) if the patient has mild renal impairment, administering about 1 to about 12 sprays per day of an oromucosal comprising THC and CBD. In embodiments, the patient with mild renal impairment is administered about 2.5 mg to about 32.4 mg of THC and about 2.5 mg to about 32.4 mg of CBD per day. In embodiments, the patient with moderate renal impairment is administered about 2.5 mg to about 16.2 mg of THC and about 2.5 mg to about 16.2 mg of CBD per day. In embodiments, the patient with severe renal impairment is administered about 2.5 mg to about 16.2 mg of THC and about 2.5 mg to about 16.2 mg of CBD per day. In some embodiments, the patient with severe renal impairment has end stage renal disease (ESRD). In some embodiments, the patient with ESRD does not receive dialysis.

In embodiments, the patient is administered the oromucosal spray in combination with one or more anti-spasticity medications. In embodiments, the one or more anti-spasticity medications is baclofen, tizanidine, dantrolene sodium, diazepam, clonazepam, and gabapentin.

In embodiments, the patient with advanced cancer has moderate to severe pain. In embodiments, the patient experiences moderate to severe pain during the highest tolerated of a strong opioid therapy. In embodiments, the strong opioid is tramadol, buprenorphine, methadone, diamorphine, fentanyl, hydromorphone, morphine, oxycodone, or pethidine.

In embodiments, the patient is an adult. In embodiments, the patient is 18-65 years old.

In embodiments, the oromucosal spray comprises a botanical drug substance comprising THC and CBD. In embodiments, the oromucosal spray further comprises one or more cannabinoids in addition to THC and CBD. In embodiments, the one or more cannabinoids are cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof. In embodiments, the oromucosal spray further comprises one or more terpenes. In embodiments, the oromucosal spray further comprises one or more sesquiterpenes. In embodiments, the one or more terpenes or sesquiterpenes are beta-farnesene, selina-3,7(11)-diene, guaia-3,9-diene, trans-caryophyllene, alpha-caryophyllene, trans-nerolidol, myrcene, trans-phytol, squalene, alpha-tocopherol, or a combination thereof. In embodiments, the oromucosal spray further comprise one or more sterols. In embodiments, the one or more sterols are beta-sitosterol, beta-amyrin, campesterol, lupeol, or combinations thereof.

### BRIEF DESCRIPTION OF THE DRAWINGS

FIGS. 1A-B provide plots representing the mean plasma concentrations of THC (FIG. 1A) and the mean logarithmic plasma concentrations of THC (FIG. 1B) versus time following administration of an oromucosal spray comprising THC and CBD (4 sprays) to human subjects with normal renal function (•) or severe renal impairment (□).

FIGS. 2A-B provide plots representing the mean plasma concentrations of CBD (FIG. 2A) and the mean logarithmic plasma concentrations of CBD (FIG. 2B) versus time following administration of an oromucosal spray comprising THC and CBD (4 sprays) to human subjects with normal renal function (•) or severe renal impairment (□).

FIGS. 3A-B provide plots representing the mean plasma concentrations of 11-OH-THC (FIG. 3A) and the mean logarithmic plasma concentrations of 11-OH-THC (FIG. 3B) versus time following administration of an oromucosal spray comprising THC and CBD (4 sprays) to human subjects with normal renal function (•) or severe renal impairment (□).

FIGS. 4A-B provide plots representing the mean plasma concentrations of 11-COOH-THC (FIG. 4A) and the mean logarithmic plasma concentrations of 11-COOH-THC (FIG. 4B) versus time following administration of an oromucosal spray comprising THC and CBD (4 sprays) to human subjects with normal renal function (•) or severe renal impairment (□).

FIGS. 5A-B provide plots representing mean plasma concentrations of 7-COOH-CBD (FIG. 5A) and the mean logarithmic plasma concentrations of 7-COOH-CBD (FIG. 5B) versus time following administration of an oromucosal spray comprising THC and CBD (4 sprays) to human subjects with normal renal function (•) or severe renal impairment (a).

FIGS. 6A-B provide plots representing mean plasma concentrations of 7-OH-CBD (FIG. 6A) and the mean logarithmic plasma concentrations of 7-OH-CBD (FIG. 6B) versus time following administration of an oromucosal spray comprising THC and CBD (4 sprays) to human subjects with normal renal function (•) or severe renal impairment (□).

FIGS. 7A-7F show the log-transformed maximum concentration (Cₘₐₓ) versus eGFR profiles of THC (FIG. 7A), CBD (FIG. 7B), 11-OH-THC (FIG. 7C), 11-COOH-THC (FIG. 7D), 7-COOH-CBD (FIG. 7E), and 7-OH-CBD (FIG. 7F) following administration of an oromucosal spray (4 sprays) to subjects with normal renal function (•) or severe renal impairment (□).

FIGS. 8A-8F show log-transformed AUC₀₋ₜₗₐₛₜ versus eGFR profiles of THC (FIG. 7A), CBD (FIG. 7B), 11-OH-THC (FIG. 7C), 11-COOH-THC (FIG. 7D), 7-COOH-CBD (FIG. 7E), and 7-OH-CBD (FIG. 7F) following administration of an oromucosal spray to subjects with normal renal function (•) or severe renal impairment (□).

### DETAILED DESCRIPTION OF THE INVENTION

### Definitions

The term “a” or “an” refers to one or more of that entity; for example, “a cannabinoid” refers to one or more cannabinoids or at least one cannabinoid. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

The term “about” refers to an acceptable degree of variation in the art. In embodiments, “about” means plus or minus 10% of the referenced number unless otherwise stated or otherwise evident by the context, and except where such a range would exceed 100% of a possible value, or fall below 0% of a possible value, such as less than 0% content of an ingredient, or more than 100% of the total contents of a composition.

The terms “oromucosal spray”, “buccal spray”, and “spray” are used interchangeably herein, and refers to a pharmaceutical formulation comprising cannabinoids that is administered by spraying into the oral mucosa, which may include, but is not limited to, the oral cavity and/or the pharynx.

The term “otherwise identical patient” or an “otherwise identical patient who does not have renal impairment” refers to a patient whose physical characteristics relevant to cannabinoids dosing are expected to be substantially the same as that of the patient with renal impairment. In embodiments, the otherwise identical patient will be of substantially the same age, sex, and body weight. In embodiments, the substantially identical patient will also have substantially identical hepatic function and drug metabolism.

The term “renal impairment” refers to a condition in which the kidney is unable to perform its usual function of removing waste or extra water from the blood or keeping bodily chemical components in balance. Renal impairment may be characterized as “mild,” “moderate,” or “severe” depending on the estimated glomerular filtration rate [eGFR]. “Normal renal function” is characterized by an [eGFR] equal to or greater than 90 mL/min/1.73 m². “Mild renal impairment” is characterized by an [eGFR] between 60 and 89 mL/min/1.73 m². “Moderate renal impairment” is characterized by an [eGFR] between 30 and 59 mL/min/1.73 m². “Severe renal impairment” is characterized by an [eGFR] below 30 mL/min/1.73 m². A patient is considered to have “end stage renal disease (ESRD)” when the [eGFR] is below 15 mL/min/1.73 m².

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “botanical drug substance” refers to a drug substance (active agent or API) comprised of plant materials.

### Discovery of Problem and Solution

Patients with normal renal function may be safely and effectively treated with a maximum of 12 sprays of the oromucosal spray described herein comprising therapeutically effective amounts of Δ-9-tetrahydrocannabinol (THC) and cannabidiol (CBD) (e.g., in combination with other cannabinoids, terpenes and sterols. Prior to the present application, there have been no studies evaluating the impact of impaired renal function on safety and efficacy of the oromucosal spray described herein.

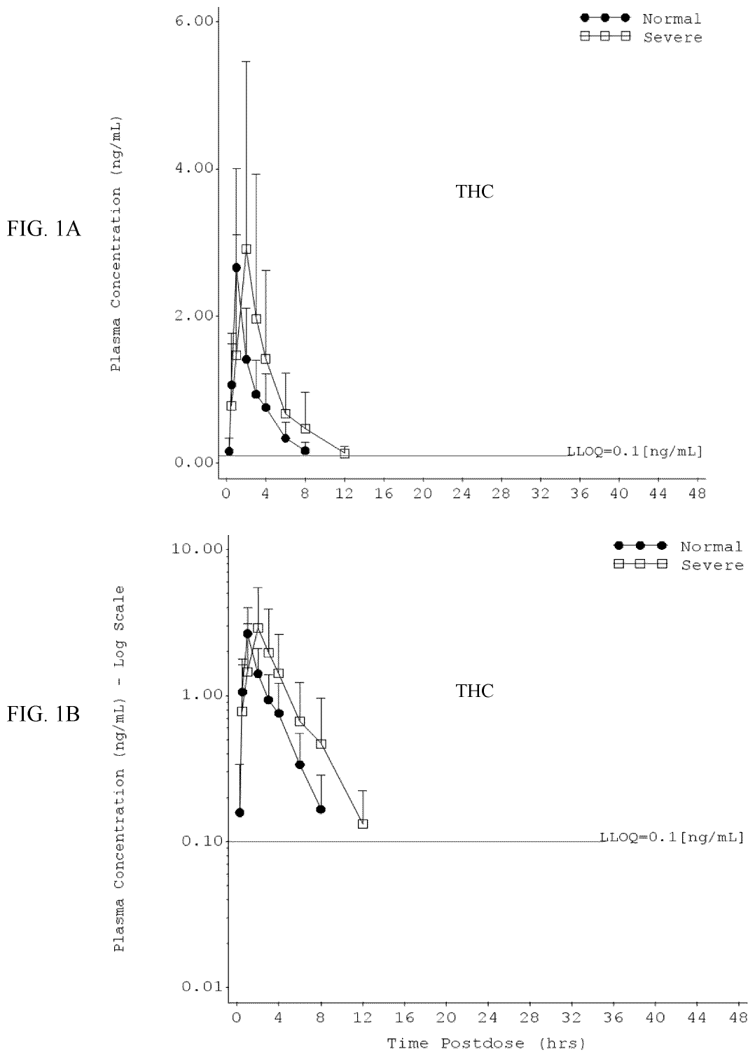

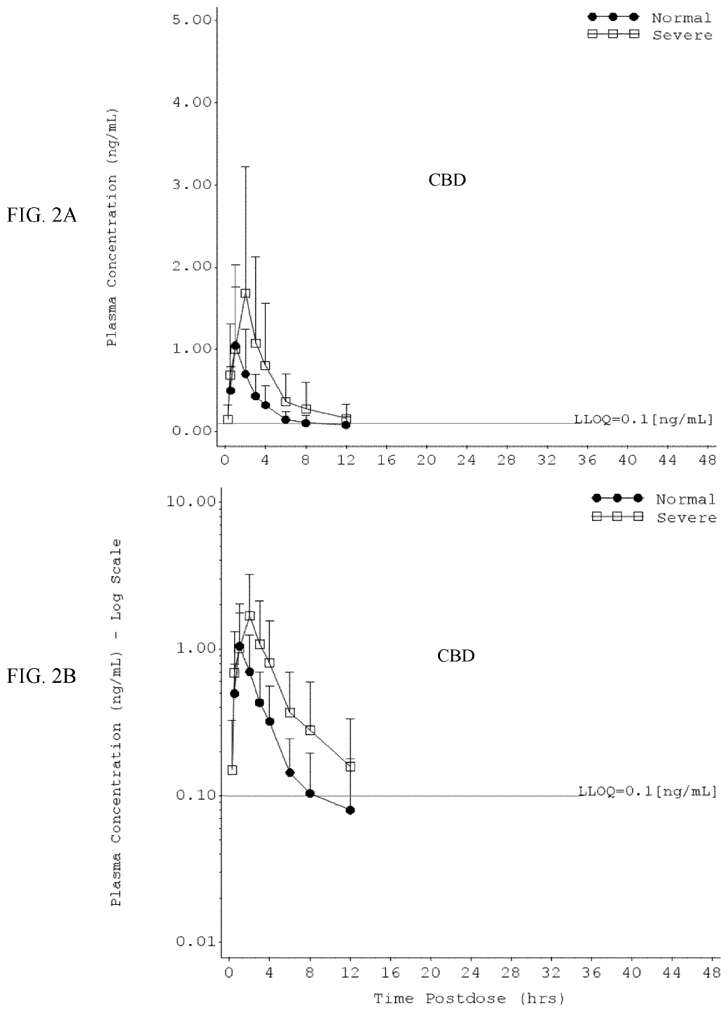

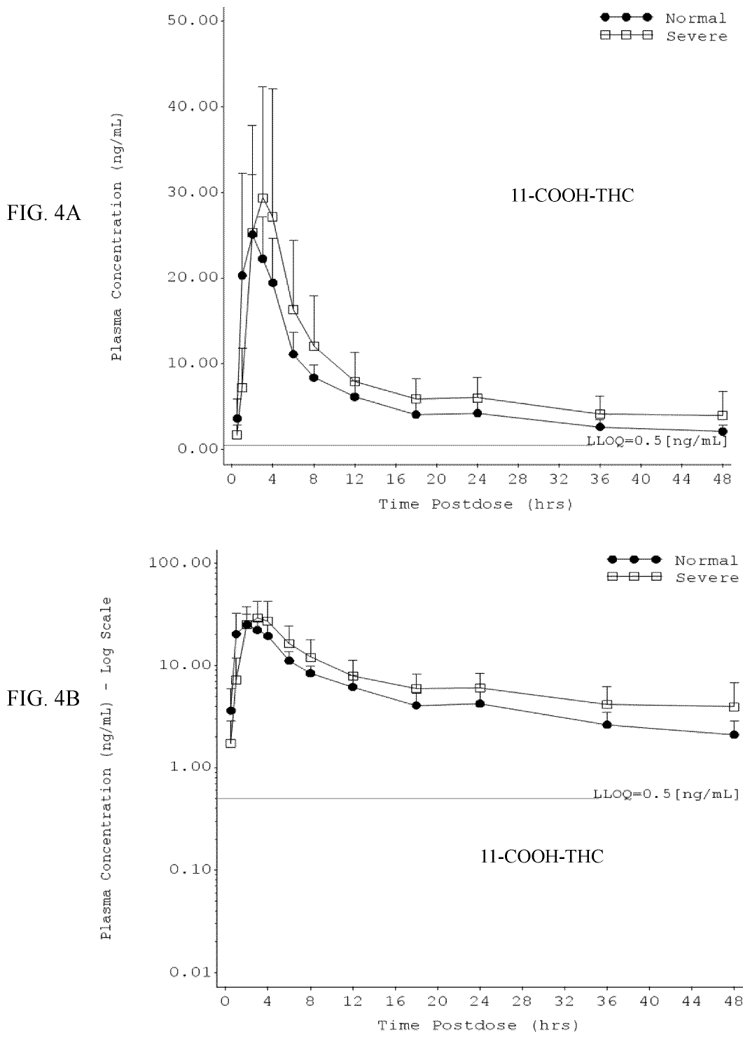

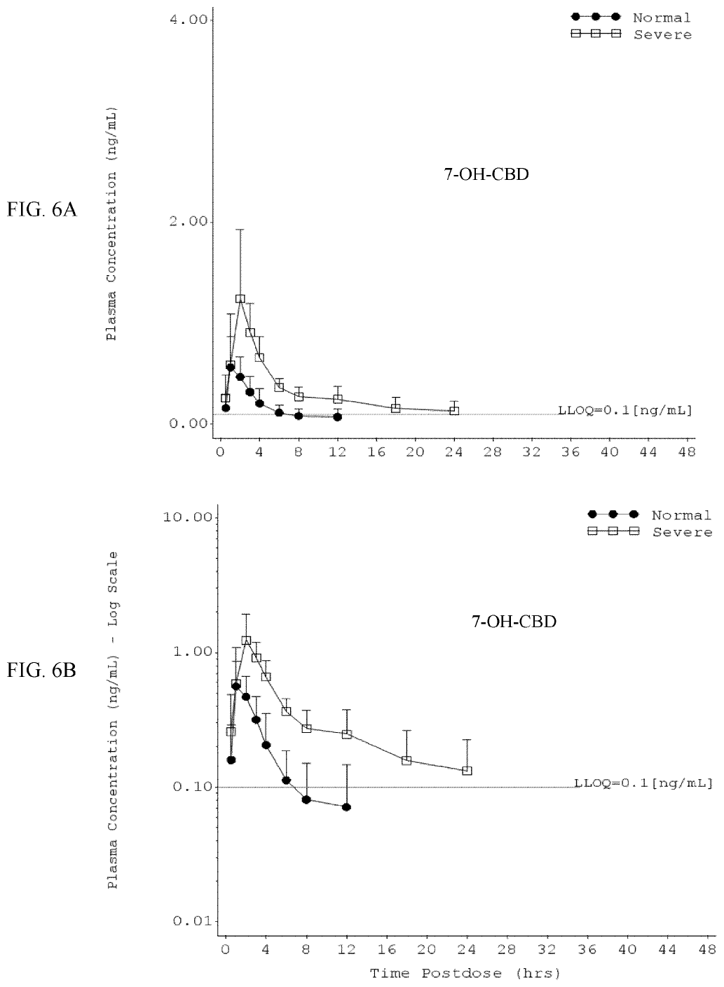

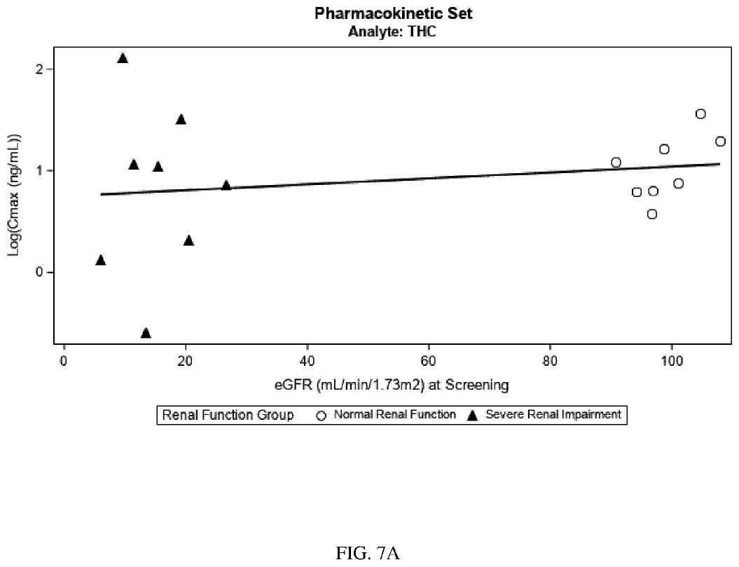

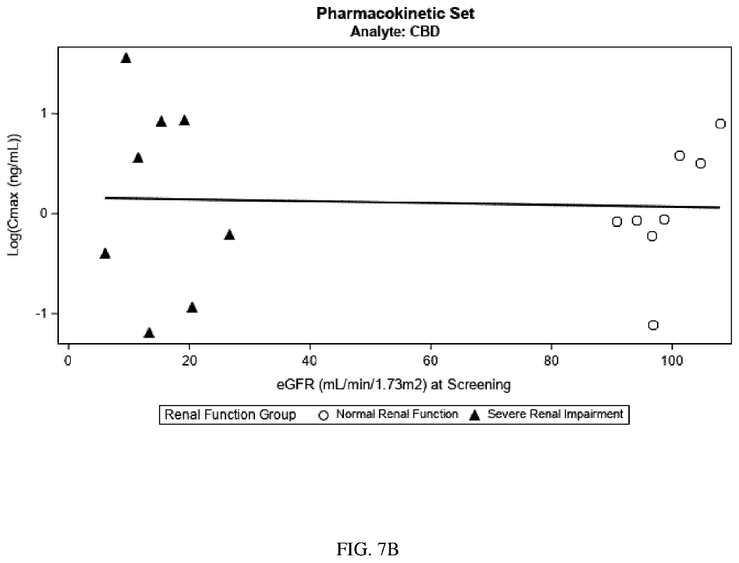

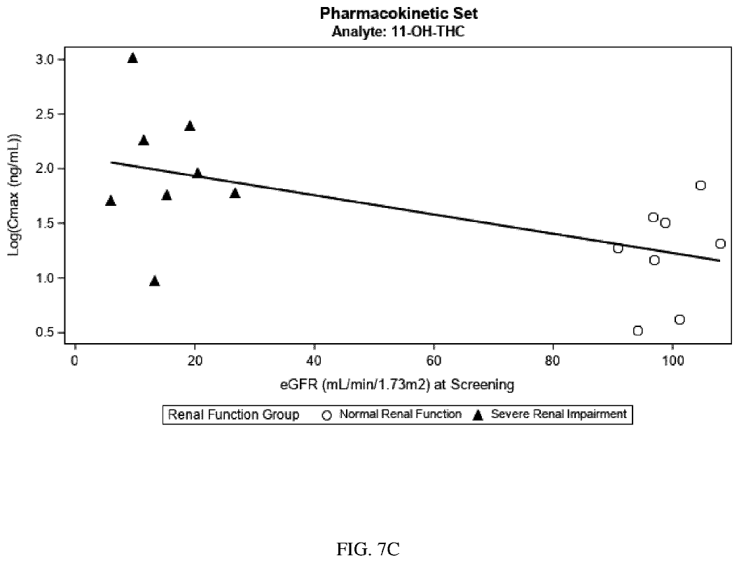

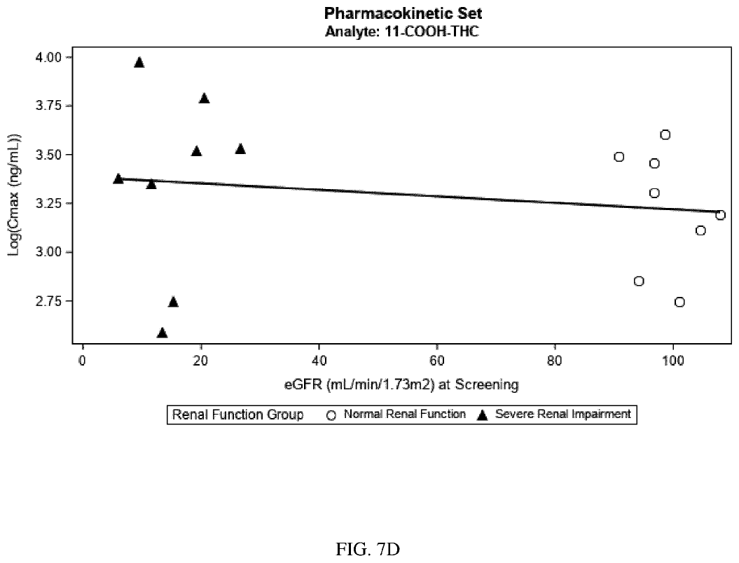

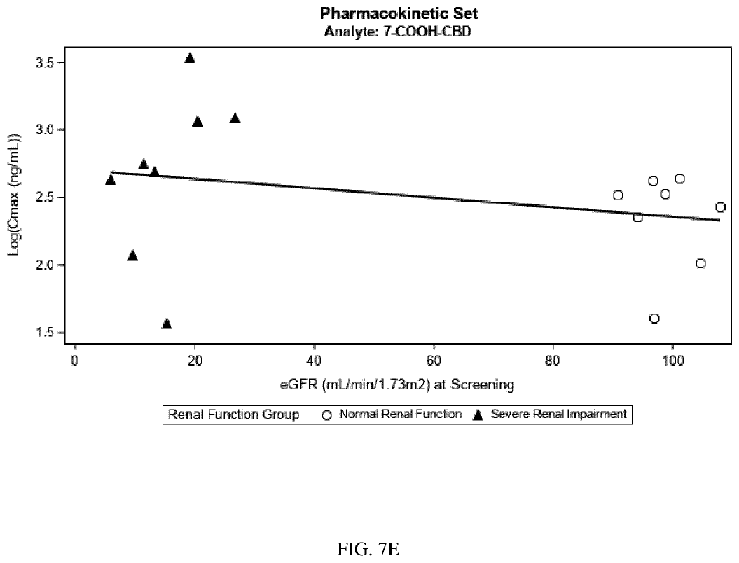

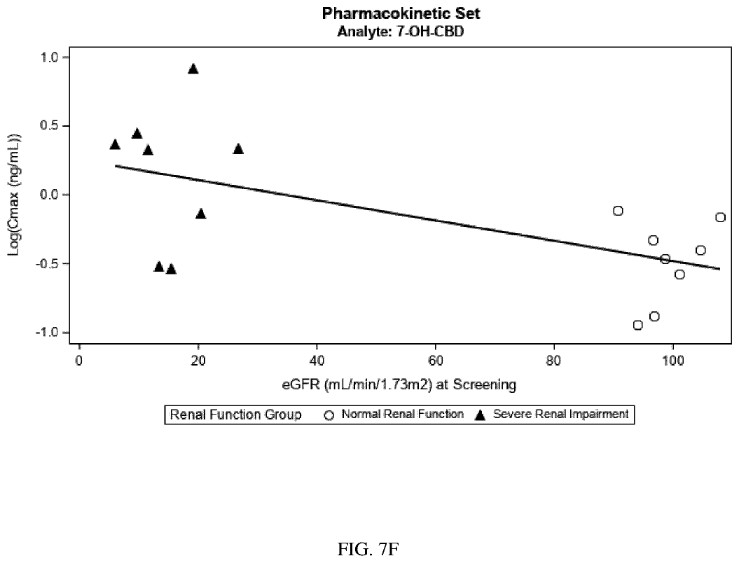

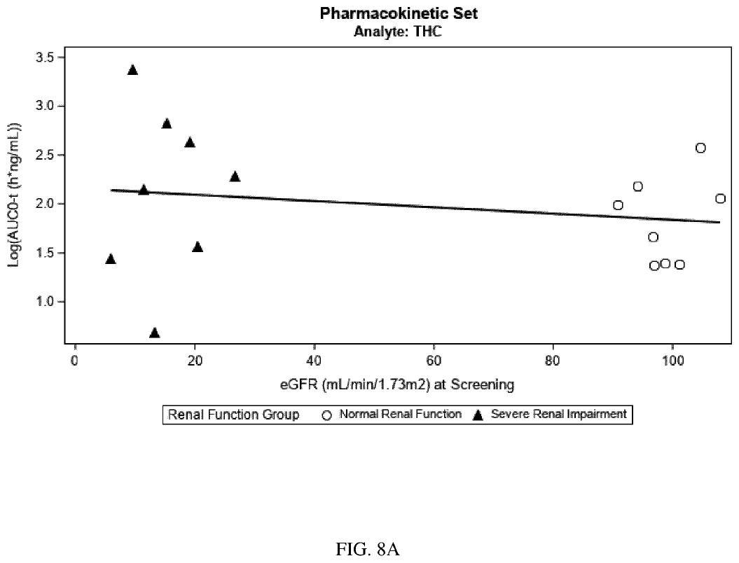

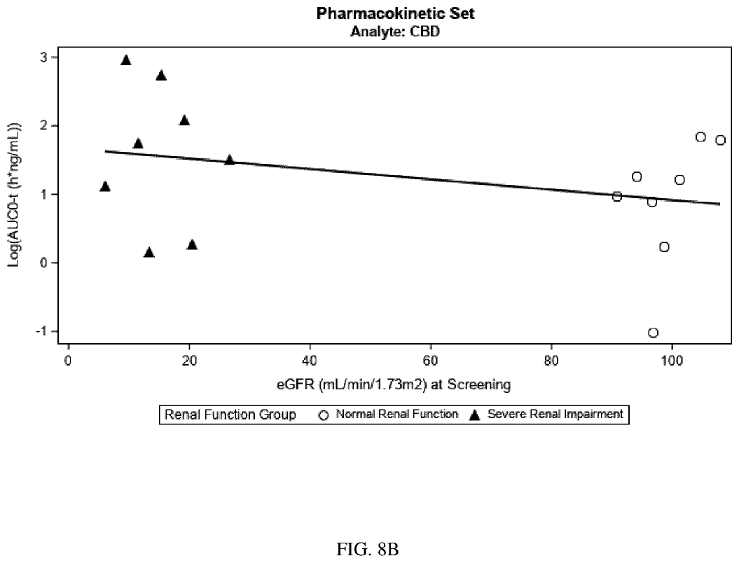

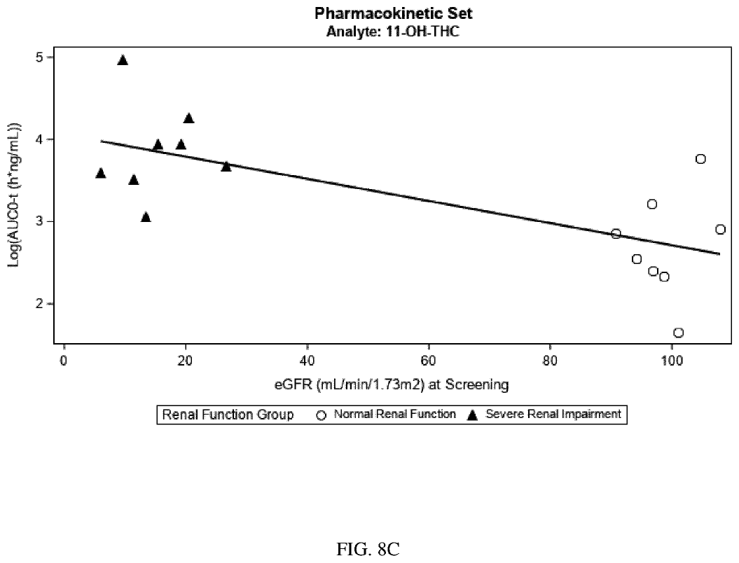

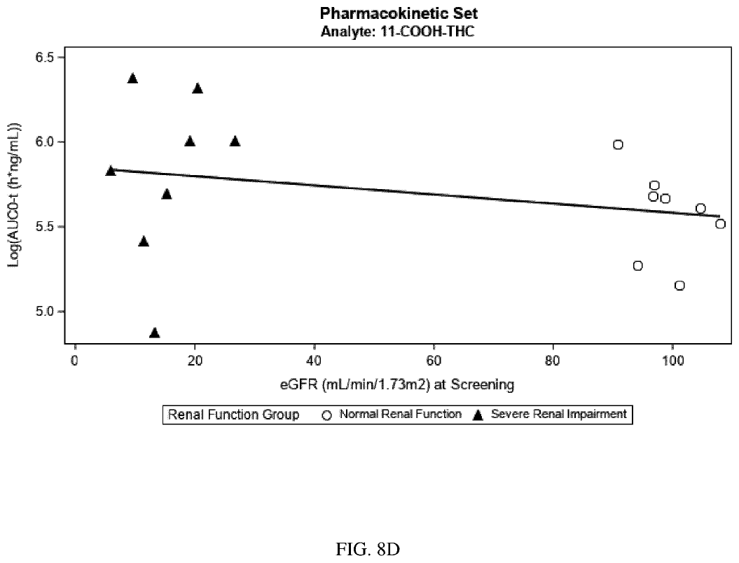

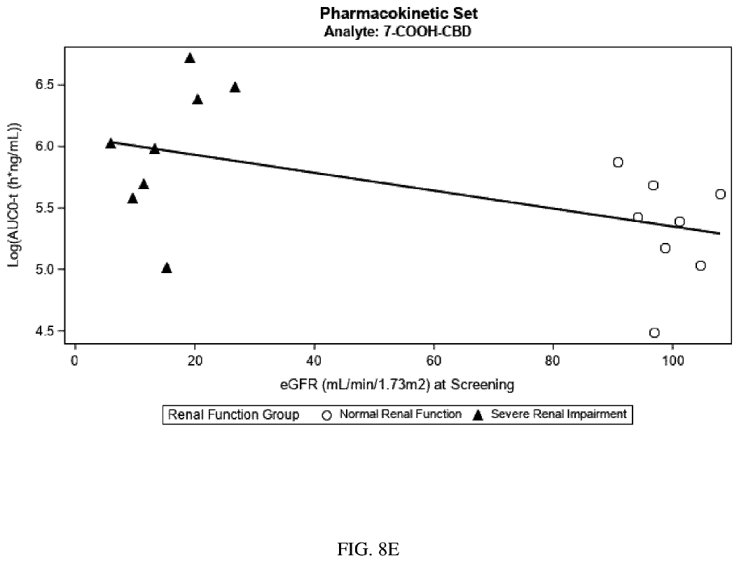

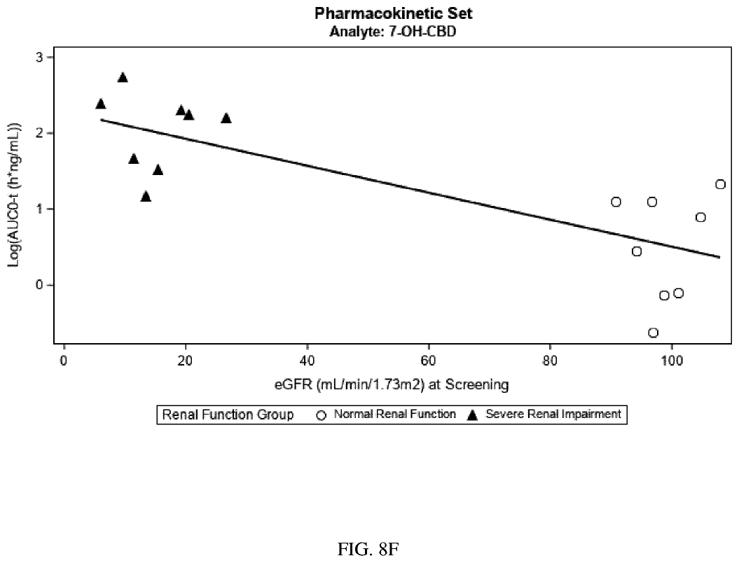

As discussed herein, the present disclosure relates to methods for safely and effectively treating spasticity or neuropathic pain in renally impaired patients with MS, and pain in renally impaired patients with advanced cancer. MS spasticity, spasticity associated with MS, and spasticity due to MS may be used interchangeably herein. Similarly, MS neuropathic pain, MS pain, pain associated with MS, and pain due to MS may be used interchangeably herein. In embodiments, the renally impaired patient has mild, moderate, or severe renal impairment. The methods of the disclosure are based on Applicant's discovery that plasma levels of cannabinoids and their metabolites are elevated in patients with varying degrees of renal impairment following administration of the oromucosal spray described herein. More specifically, as described in the Examples, Applicant observed clinically significant elevations of plasma levels of pharmacologically active metabolites of THC and CBD (e.g., 11-OH-THC and 7-OH-CBD) in patients with renal impairment but not the parent compound THC and CBD, or carboxylic acid metabolites (7-COOH CBD and 11-COOH THC. As shown in FIGS. 1-6, Applicant discovered that severe renal impairment results in up to about 1.95-fold increase in the mean plasma concentrations over time (AUCₗₐₛₜ) for parent cannabinoid molecules (i.e., THC and CBD) and the carboxylic acid metabolites (i.e., 7-COOH CBD and 11-COOH THC), while their pharmacologically active hydroxylated metabolites (7-OH CBD and 11-OH THC) exhibited 3- to 5-fold elevations in AUCₗₐₛₜ. Additionally, Applicant observed clinically significant prolonged elimination of 11-OH-THC, 7-OH-CBD in patients with renal impairment, while elimination of the parent cannabinoids THC and CBD and the carboxylic acid metabolites (7-COOH CBD and 11-COOH THC) were relatively unchanged. Increased plasma concentrations and prolonged elimination of hydroxy metabolites (11-OH THC and 7-OH CBD), were notably greater than that of the parent molecules or the carboxylic acid-metabolites (7-COOH CBD and 11-COOH THC), suggesting that there is an unexpectedly differential and clinically relevant impact on metabolism and excretion of the hydroxy metabolites over the parent molecules or the carboxylic acid metabolites in patients with renal impairment.

These results are also surprising when compared with the results of similar studies in renally impaired patients that were administered a highly purified form of CBD from botanical origin. The highly purified form of CBD refers to a CBD drug substance comprising at least 99% CBD and less than 0.15% THC. In contrast to the results described herein, clinically relevant elevations of CBD, or the 7-OH-CBD and 7-COOH-CBD-metabolites (e.g., mean AUCₗₐₛₜ ratios greater than 2 between the renally impaired and the control) were not observed when the highly purified form of CBD was administered to renally impaired patients. Furthermore, the different THC to CBD ratio in the oral mucosal spray described herein versus the highly purified CBD was not expected to affect the disposition of CBD or THC based on their metabolic properties. Thus, this data indicates that the oromucosal spray comprising a combination of THC and CBD at the amounts and ratios disclosed herein (e.g., prepared from Cannabis extracts) cause an unexpected systemic disposition of cannabinoid metabolism and/or excretion in patients with renal impairment that is not generally observed with other cannabinoid compositions.

### Patient Populations

The methods of this disclosure are used to treat patients with varying degrees of renal impairment who are suffering from neurological pain, long-term pain associated with cancer, or MS-associated spasticity. Renal impairment in those patients can be mild, moderate, or severe.

In embodiments, the patients described herein have severe renal impairment. In embodiments, the patients have End-Stage Renal Disease (ESRD). In embodiments, the patient with ESRD does not require dialysis. In embodiments, the patient with severe renal impairment or ESRD does not receive dialysis treatment.

In embodiments, the patient with mild, moderate, or severe renal impairment does not have hepatic impairment. In embodiments, the patient with renal impairment does not have any history or clinical manifestation of any metabolic, allergic, dermatological, hepatic, hematological, pulmonary, cardiovascular, gastrointestinal, neurological, respiratory, endocrine, or psychiatric disorder, or any other significant disease or disorder.

In embodiments, the patient is an adult. In embodiments, the patient 18-90 years old, e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90. In embodiments, the patient is an adult but not elderly. In embodiments, the patient is 18-65 years old. In embodiments, the patient can be >65 years old, such as 66-90 years old.

### Methods of Treatment

As discussed herein, the amount of the oromucosal spray administered depends on the renal status of the patient. In embodiments, patients with moderate or severe renal impairment may receive a reduced dose of the oromucosal spray. In embodiments, patients with moderate or severe renal impairment are administered a lower maximum dose. The reduced dose is less than the dose that an otherwise identical patient with normal renal function would receive. In other words, the reduced dose of cannabinoids administered to a patient with a certain degree of renal impairment is less than the dose of cannabinoids administered to the same (or similar) patient with normal kidney function. A similar patient is a patient of approximately the same age, gender, and disease severity. In embodiments, the number of sprays administered to a patient with moderate or severe renal impairment may be reduced by 1, 2, 3, 4, 5, 6, 7, 8, 9, 10, or 11 sprays, including any value or range therein, compared to the dose of cannabinoids administered to the same (or similar) patient with normal kidney function. In embodiments, the number of sprays administered to a patient with moderate or severe renal impairment may be reduced by about 5%, 10%, 15%, 20%, 25%, 30%, 35%, 40%, 45%, 50%, 55%, 60%, 65%, 70%, 75%, 80%, 85%, 90%, 95%, or 99%, including any value or range therein, compared to the dose of cannabinoids administered to the same (or similar) patient with normal kidney function.

In embodiments, the disclosure provides a method of treating or managing spasticity in a patient with multiple sclerosis, wherein the patient has moderate or severe renal impairment. In embodiments, the patient with moderate or severe renal impairment is administered a maximum of 6 sprays per day of an oromucosal spray comprising Δ-9-tetrahydrocannabinol (THC) and cannabidiol (CBD). In embodiments, the patient with moderate or severe renal impairment is administered 1 to 6 sprays per day of the oromucosal spray. In embodiments, the patient with moderate or severe renal impairment is administered 1 spray per day of the oromucosal spray. In embodiments, the patient with moderate or severe renal impairment is administered 2 sprays per day of the oromucosal spray. In embodiments, the patient with moderate or severe renal impairment is administered 3 sprays per day of the oromucosal spray. In embodiments, the patient with moderate or severe renal impairment is administered 4 sprays per day of the oromucosal spray. In embodiments, the patient with moderate or severe renal impairment is administered 5 sprays per day of the oromucosal spray. In embodiments, the patient with moderate or severe renal impairment is administered 6 sprays per day of the oromucosal spray. In some embodiments, the patient with severe renal impairment has end stage renal disease (ESRD). In some embodiments, the patient with ESRD does not receive dialysis. In embodiments, the patient with multiple sclerosis has moderate to severe spasticity. Methods of determining the severity of spasticity are known in the art. See, e.g., Hugos and Cameron, Curr. Neurol. Neurosci. Rep. 2019 Aug. 30; 19 (10): 79. In embodiments, the patient has not responded adequately to other anti-spasticity medication. In embodiments, the oromucosal spray is administered in combination (e.g., as an adjunctive treatment) with anti-spasticity medication. Non-limiting examples of anti-spasticity include baclofen (Lioresal®), tizanidine (Zanaflex®), dantrolene sodium (Dantrium®), diazepam (Valium®), clonazepam (Klonopin®), and gabapentin (Neurontin®). In embodiments, the anti-spasticity medication may be administered sequentially, concurrently, or simultaneously with the oromucosal spray.

In embodiments, the disclosure provides a method of treating or managing spinal cord injury spasticity in a patient, wherein the patient has moderate or severe renal impairment. In embodiments, the patient with moderate or severe renal impairment is administered a maximum of 6 sprays per day of an oromucosal spray comprising Δ-9-tetrahydrocannabinol (THC) and cannabidiol (CBD). In embodiments, the patient with moderate or severe renal impairment is administered 1 to 6 sprays per day of the oromucosal spray. In embodiments, the patient with moderate or severe renal impairment is administered 1 spray per day of the oromucosal spray. In embodiments, the patient with moderate or severe renal impairment is administered 2 sprays per day of the oromucosal spray. In embodiments, the patient with moderate or severe renal impairment is administered 3 sprays per day of the oromucosal spray. In embodiments, the patient with moderate or severe renal impairment is administered 4 sprays per day of the oromucosal spray. In embodiments, the patient with moderate or severe renal impairment is administered 5 sprays per day of the oromucosal spray. In embodiments, the patient with moderate or severe renal impairment is administered 6 sprays per day of the oromucosal spray. In some embodiments, the patient with severe renal impairment has end stage renal disease (ESRD). In some embodiments, the patient with ESRD does not receive dialysis. In embodiments, the patient with spinal cord injury has moderate to severe spasticity. Methods of determining the severity of spasticity are known in the art. See, e.g., Hugos and Cameron, Curr. Neurol. Neurosci. Rep. 2019 Aug. 30; 19 (10): 79. In embodiments, the patient has not responded adequately to other anti-spasticity medication. In embodiments, the oromucosal spray is administered in combination (e.g., as an adjunctive treatment) with anti-spasticity medication. Non-limiting examples of anti-spasticity include baclofen (Lioresal®), tizanidine (Zanaflex®), dantrolene sodium (Dantrium®), diazepam (Valium®), clonazepam (Klonopin®), and gabapentin (Neurontin®). In embodiments, the anti-spasticity medication may be administered sequentially, concurrently, or simultaneously with the oromucosal spray.

In embodiments, a method of treating neuropathic pain associated with multiple sclerosis in a patient with moderate or severe renal impairment. In embodiments, the patient with moderate or severe renal impairment is administered a maximum of 6 sprays per day of an oromucosal spray comprising THC and CBD. In embodiments, the patient with moderate or severe renal impairment is administered 1 to 6 sprays per day of an oromucosal spray. In embodiments, the patient with moderate or severe renal impairment is administered 1 spray per day of the oromucosal spray. In embodiments, the patient with moderate or severe renal impairment is administered 2 sprays per day of the oromucosal spray. In embodiments, the patient with moderate or severe renal impairment is administered 3 sprays per day of the oromucosal spray. In embodiments, the patient with moderate or severe renal impairment is administered 4 sprays per day of the oromucosal spray. In embodiments, the patient with moderate or severe renal impairment is administered 5 sprays per day of the oromucosal spray. In embodiments, the patient with moderate or severe renal impairment is administered 6 sprays per day of the oromucosal spray. In embodiments, the oromuoscsal spray is administered in combination with (e.g., as an adjunctive treatment) medications used to treat neuropathic pain. Non-limiting examples of medications used to treat neuropathic pain include tricyclic antidepressants (TCAs), serotonin/norepinephrine reuptake inhibitors (SSRIs), and some anticonvulsants, for example, gabapentin or topical lidocaine. Non-limiting examples of TCAs include doxepin, imipramine, amitriptyline, nortriptyline and desipramine. Non-limiting examples of SSRIs include paroxetine and citalopram. In some embodiments, the oromucosal spray is administered in combination with an opioid analgesic (e.g., morphine, oxycodone, methadone, and fentanyl) or antiepileptic drugs (e.g., carbamazepine, lamotrigine, oxcarbazepine, topiramate, and valproic acid), mexiletine, and topical capsaicin.

In embodiments, a method of treating pain in a patient with advanced cancer and having moderate or severe renal impairment. In embodiments, the patient with moderate or severe renal impairment is administered a maximum of 6 sprays per day of an oromucosal spray comprising THC and CBD. In embodiments, the patient is administered 1 to 6 sprays per day of the oromucosal spray. In embodiments, the patient with moderate or severe renal impairment is administered 1 spray per day of the oromucosal spray. In embodiments, the patient with moderate or severe renal impairment is administered 2 sprays per day of the oromucosal spray. In embodiments, the patient with moderate or severe renal impairment is administered 3 sprays per day of the oromucosal spray. In embodiments, the patient with moderate or severe renal impairment is administered 4 sprays per day of the oromucosal spray. In embodiments, the patient with moderate or severe renal impairment is administered 5 sprays per day of the oromucosal spray. In embodiments, the patient with moderate or severe renal impairment is administered 6 sprays per day of the oromucosal spray. In embodiments, the patient with advanced cancer experiences moderate to severe pain. Pain scales are known in the art, such as the Numerical Rating Scales (NRS), Visual Analog Scales (VAS), and Categorical Scales. Further details on response scales are provided in Hjermstad, M. J. et al. J. Pain Symptom Manag. 2011, 41, 1073-1093 and Yang et al. Sustainability 2019, 11, 3975, the contents of which are herein incorporated by reference in its entirety. For example, the Numerical Rating Pain Scale scores pain from 1 to 10. Scores from 4-6 indicate moderate pain. Scores from 7-10 indicate severe pain. In embodiments, the patient experiences moderate to severe pain during the highest tolerated of a strong opioid therapy. In embodiments, the strong opioid is administered for persistent background pain. In embodiments, the oromucosal spray is administered in combination with a strong opioid. Non-limiting examples of strong opioids include tramadol, buprenorphine, methadone, diamorphine, fentanyl, hydromorphone, morphine, oxycodone, and pethidine. In embodiments, the strong opioid may be administered sequentially, concurrently, or simultaneously with the oromucosal spray.

In embodiments, the patient with advanced cancer has severe renal impairment. In embodiments, the patient is administered a maximum of about 6 sprays per day of an oromucosal spray comprising THC and CBD. In embodiments, the patient with advanced cancer having severe renal impairment is administered 1 spray per day of the oromucosal spray. In embodiments, the patient with advanced cancer having severe renal impairment is administered 2 sprays per day of the oromucosal spray. In embodiments, the patient with advanced cancer having severe renal impairment is administered 3 sprays per day of the oromucosal spray. In embodiments, the patient with advanced cancer having severe renal impairment is administered 4 sprays per day of the oromucosal spray. In embodiments, the patient with advanced cancer having severe renal impairment is administered 5 sprays per day of the oromucosal spray. In embodiments, the patient with advanced cancer having severe renal impairment is administered 6 sprays per day of the oromucosal spray. In some embodiments, the patient with severe renal impairment has end stage renal disease (ESRD). In some embodiments, the patient with ESRD does not receive dialysis.

In embodiments, the patient with advanced cancer has moderate renal impairment. In embodiments, the patient with advanced cancer having moderate renal impairment is administered a maximum of 1 spray per day of an oromucosal spray comprising THC and CBD. In such embodiments, the patient with advanced cancer having moderate renal impairment is administered 1 spray per day of the oromucosal spray. In embodiments, the patient with advanced cancer having moderate renal impairment is administered 2 sprays per day of the oromucosal spray. In embodiments, the patient with advanced cancer having moderate renal impairment is administered 3 sprays per day of the oromucosal spray. In embodiments, the patient with advanced cancer having moderate renal impairment is administered 4 sprays per day of the oromucosal spray. In embodiments, the patient with advanced cancer having moderate renal impairment is administered 5 sprays per day of the oromucosal spray. In embodiments, the patient with advanced cancer having moderate renal impairment is administered 6 sprays per day of the oromucosal spray.

In one aspect, the present disclosure provides a method of treating spasticity associated with multiple sclerosis in a patient with mild renal impairment. In embodiments, the patient with mild renal impairment is administered a maximum of 12 sprays per day of an oromucosal spray comprising THC and CBD. In embodiments, the patient with mild renal impairment is administered from 1 to 12 sprays per day of an oromucosal spray comprising THC and CBD. In embodiments, the patient is administered about 1 spray per day of the oromucosal spray. In embodiments, the patient with mild renal impairment is administered 2 sprays per day of the oromucosal spray. In embodiments, the patient with mild renal impairment is administered 3 sprays per day of the oromucosal spray. In embodiments, the patient with mild renal impairment is administered 4 sprays per day of the oromucosal spray. In embodiments, the patient with mild renal impairment is administered 5 sprays per day of the oromucosal spray. In embodiments, the patient with mild renal impairment is administered 6 sprays per day of the oromucosal spray. In embodiments, the patient with mild renal impairment is administered 7 sprays per day of the oromucosal spray. In embodiments, the patient with mild renal impairment is administered 8 sprays per day of the oromucosal spray. In embodiments, the patient with mild renal impairment is administered 9 sprays per day of the oromucosal spray. In embodiments, the patient with mild renal impairment is administered 10 sprays per day of the oromucosal spray. In embodiments, the patient with mild renal impairment is administered 11 sprays per day of the oromucosal spray. In embodiments, the patient with mild renal impairment is administered 12 sprays per day of the oromucosal spray.

In one aspect, a method of treating neuropathic pain associated with multiple sclerosis in a patient with mild renal impairment. In embodiments, the patient with mild renal impairment is administered a maximum of 12 sprays per day of an oromucosal spray comprising THC and CBD. In embodiments, the patient is administered from about 1 to about 12 sprays per day of an oromucosal spray comprising THC and CBD. In such embodiments, the patient with mild renal impairment is administered 1 spray per day of the oromucosal spray. In embodiments, the patient with mild renal impairment is administered 2 sprays per day of the oromucosal spray. In embodiments, the patient with mild renal impairment is administered 3 sprays per day of the oromucosal spray. In embodiments, the patient with mild renal impairment is administered 4 sprays per day of the oromucosal spray. In embodiments, the patient with mild renal impairment is administered 5 sprays per day of the oromucosal spray. In embodiments, the patient with mild renal impairment is administered 6 sprays per day of the oromucosal spray. In embodiments, the patient with mild renal impairment is administered 7 sprays per day of the oromucosal spray. In embodiments, the patient with mild renal impairment is administered 8 sprays per day of the oromucosal spray. In embodiments, the patient with mild renal impairment is administered 9 sprays per day of the oromucosal spray. In embodiments, the patient with mild renal impairment is administered 10 sprays per day of the oromucosal spray. In embodiments, the patient with mild renal impairment is administered 11 sprays per day of the oromucosal spray. In embodiments, the patient with mild renal impairment is administered 12 sprays per day of the oromucosal spray.

In one aspect, a method of treating pain in a patient with advanced cancer and having mild renal impairment. In embodiments, the patient is from about 1 to about 12 sprays per day of an oromucosal spray comprising THC and CBD. In embodiments, the patient with mild renal impairment is administered a maximum of 12 sprays per day of an oromucosal spray comprising THC and CBD. In such embodiments, the patient with mild renal impairment is administered 1 spray per day of the oromucosal spray. In embodiments, the patient with mild renal impairment is administered 2 sprays per day of the oromucosal spray. In embodiments, the patient with mild renal impairment is administered 3 sprays per day of the oromucosal spray. In embodiments, the patient with mild renal impairment is administered 4 sprays per day of the oromucosal spray. In embodiments, the patient with mild renal impairment is administered 5 sprays per day of the oromucosal spray. In embodiments, the patient with mild renal impairment is administered 6 sprays per day of the oromucosal spray. In embodiments, the patient with mild renal impairment is administered 7 sprays per day of the oromucosal spray. In embodiments, the patient with mild renal impairment is administered 8 sprays per day of the oromucosal spray. In embodiments, the patient with mild renal impairment is administered 9 sprays per day of the oromucosal spray. In embodiments, the patient with mild renal impairment is administered 10 sprays per day of the oromucosal spray. In embodiments, the patient with mild renal impairment is administered 11 sprays per day of the oromucosal spray. In embodiments, the patient with mild renal impairment is administered 12 sprays per day of the oromucosal spray.

In another aspect, the present disclosure further provides a method of managing treatment of spasticity associated with multiple sclerosis, neuropathic pain associated with multiple sclerosis, or pain in a patient with advanced cancer, in a patient with renal impairment, in which the method comprises the steps of (a) determining the patient's renal impairment status; (i) if the patient has moderate or severe renal impairment, administering a maximum of 6 sprays (e.g., from 1 to 6 sprays per day of an oromucosal comprising THC and CBD; or (ii) if the patient has mild renal impairment, administering a maximum of 12 sprays per day (e.g., from 1 to 12 sprays per day) of an oromucosal comprising THC and CBD. In embodiments, the patient with mild renal impairment is administered about 2.25 mg to about 32.4 mg of THC and about 2.25 mg to about 32.4 mg of CBD per day. In embodiments, the patient with moderate renal impairment is administered about 2.25 mg to about 16.2 mg of THC and about 2.25 mg to about 16.2 mg of CBD per day. In embodiments, the patient with severe renal impairment is administered about 2.25 mg to about 16.2 mg of THC and about 2.25 mg to about 16.2 mg of CBD per day. In some embodiments, the patient with severe renal impairment has end stage renal disease (ESRD). In some embodiments, the patient with ESRD does not receive dialysis.

Table 1 lists THC/CBD dosage for patients with mild, moderate, severe renal impairment.

TABLE 1
Example Dosages for Patients with Renal Impairment

| | | | Maximum |
|---|---|---|---|
| | | | Recommended |
| Renal Impairment | Starting Dosage | Usual Dosage | Dosage |
| Mild | 1 spray (2.7 mg THC | 4-8 sprays (10.8-21.6 | 12 sprays (32.4 mg |
| | and 2.5 mg CBD) | mg THC and 10.0- | THC and 30.0 mg |
| | daily | 20.0 mg CBD) daily | CBD) daily |
| Moderate | 1 spray (2.7 mg THC | 2-4 sprays (5.4-10.8 | 6 sprays (16.2 mg |
| | and 2.5 mg CBD) | mg THC and 5.0-10.0 | THC and 15.0 mg |
| | daily | mg CBD) daily | CBD) daily |
| Severe | 1 spray (2.7 mg THC | 2-4 sprays (5.4-10.8 | 6 sprays (16.2 mg |
| | and 2.5 mg CBD) | mg THC and 5.0-10.0 | THC and 15.0 mg |
| | daily | mg CBD) daily | CBD) daily |

In embodiments, a patient with severe or moderate renal impairment is administered a maximum dose equivalent to about half of the maximum dose for a patient with normal renal function. This dose modification provides safe and therapeutically effective plasma levels of THC and CBD, as well as metabolites of THC and CBD.

In embodiments, the methods disclosed herein comprising a time interval or “gap” between administering each spray. In embodiments, the gap between sprays is at least 15 minutes. In embodiments, the gap is between sprays is 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 90 minutes, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, 24 hours, or any time intervals or ranges in between. In embodiments, the initiation spray dosing is evenly spread out throughout the day.

In embodiments, the disclosure provides a titration dosing schedule for initiating treatment. During treatment initiation, the patient starts treatment with a “starting dose” and then, over a period of time, the dose is increased to a usual or maximum dose. In embodiments, the titration schedule for initiating treatment comprises starting treatment with 1 spray per day of the oromucosal spray on Day 1 of treatment. This may be referred to as the “starting dose.” In embodiments, the starting dose of 1 spray per day is administered in the morning or at nighttime. In embodiments, the starting dose of 1 spray per day is administered at nighttime. In embodiments, the patient gradually increases the total number of sprays from the starting dose. In embodiments, the starting dose is increased by 1 spray after 1, 2, 3, or 4 days. In embodiments, the starting dose is increased after 2 days. In embodiments, the starting dose is increased by one or more sprays per day (e.g., 2, 3, or 4 sprays per day). In embodiments, the starting dose is increased by 1 spray per day. In embodiments, the number of sprays administered during treatment initiation is increased every day, every 2 days, every 3 days, every 4 days, every 5 days, every 6 days, every 7 days, or a combination thereof. In embodiments, the number of sprays administered during treatment initiation is increased every day, every 2 days, or a combination thereof. In embodiments, the number of sprays administered during treatment initiation is increased every 2 days on Days 1-4 of treatment, and then every day thereafter. In embodiments, the treatment initiation period comprises a brief “drug holiday” or “off day(s)” in which the patient is not administered the oromucosal spray. In embodiments, the patient may administer the oromucosal spray every day, every other day (e.g., on day 1, day 3, day 5, etc), every 3rd day (e.g., day 1, day 4, day 7 etc.), or every 4th day, or a combination thereof, during treatment initiation. For example, in embodiments, the patient may administer 1 spray on day 1 of treatment, 0 sprays on day 2, 2 sprays day 3, 0 sprays on day 4, 2 or 3 sprays on day 5, 0 sprays on day 6, and so on.

In embodiments, the treatment initiation occurs over 14 days. In embodiments, the duration of treatment initiation is adjusted based on the renal impairment status of the patient. In embodiments, the treatment initiation period may be less than 14 days, for example, 13 days, 12 days, 11 days, 10 days, 9 days, 8 days, or 7 days. In embodiments, the dose is increased every day, every other day, every 3 days, or every 4 days during the titration.

In embodiments where the patient is administered two or more sprays per day, the two or more sprays may be administered at nighttime or in the morning, or a combination thereof. In embodiments, the “gap” or time interval between administering the two sprays may be, for example, at least 15 minutes, e.g., 15 minutes, 20 minutes, 25 minutes, 30 minutes, 35 minutes, 40 minutes, 45 minutes, 50 minutes, 55 minutes, 60 minutes, 90 minutes, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, 24 hours, or any time intervals or ranges in between. An example titration schedule for a patient with mild renal impairment is shown in Table 2. An example titration schedule for patients with moderate or severe renal is shown in Table 3.

TABLE 2
Example Titration Period for Patient with Mild Renal Impairment

| | Number of Sprays | Number of sprays | Total number of |
|---|---|---|---|
| Day | in the Morning | in the evening | sprays per day |
| 1 | 0 | 1 | 1 |
| 2 | 0 | 1 | 1 |
| 3 | 0 | 2 | 2 |
| 4 | 0 | 2 | 2 |
| 5 | 1 | 2 | 3 |
| 6 | 1 | 3 | 4 |
| 7 | 1 | 4 | 5 |
| 8 | 2 | 4 | 6 |
| 9 | 2 | 5 | 7 |
| 10 | 3 | 5 | 8 |
| 11 | 3 | 6 | 9 |
| 12 | 4 | 6 | 10 |
| 13 | 4 | 7 | 11 |
| 14 | 5 | 7 | 12 |

TABLE 3
Example Titration Period for Patient with
Moderate or Severe Renal Impairment

| | Number of Sprays | Number of sprays | Total number of |
|---|---|---|---|
| Day | in the Morning | in the evening | sprays per day |
| 1 | 0 | 1 | 1 |
| 2 | 0 | 1 | 1 |
| 3 | 0 | 1 | 1 |
| 4 | 0 | 1 | 1 |
| 5 | 1 | 1 | 2 |
| 6 | 1 | 1 | 2 |
| 7 | 1 | 1 | 2 |
| 8 | 1 | 2 | 3 |
| 9 | 1 | 2 | 3 |
| 10 | 2 | 2 | 4 |
| 11 | 2 | 2 | 4 |
| 12 | 2 | 3 | 5 |
| 13 | 2 | 3 | 5 |
| 14 | 3 | 3 | 6 |

In embodiments, the morning dose is administered at any time from waking up to 12 noon. In embodiments, the evening dose is administered at any time from 4 pm to bedtime.

In embodiments, the oromucosal spray is administered for a defined length of time, including the titration period (referred to herein as an “administration period”). In embodiments, the oromucosal spray is administered for at least about 14 days e.g., 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 24 weeks, or 25 weeks, or 26 weeks, or 27 weeks, or 28 weeks, or 29 weeks, or 30 weeks, or 31 weeks, or 32 weeks, or 33 weeks, or 34 weeks, or 35 weeks, or 36 weeks, or 37 weeks, or 38 weeks, or 39 weeks, or 40 weeks, or 41 weeks, or 42 weeks, or 43 weeks, or 44 weeks, or 45 weeks, or 46 weeks, or 47 weeks, or 48 weeks, or 49 weeks, or 50 weeks, or 51 weeks, or 52 weeks, or 53 weeks, or 54 weeks, or 55 weeks, or 56 weeks, or 57 weeks, or 58 weeks, or 59 weeks, or 60 weeks, or 61 weeks, or 62 weeks, or 63 weeks, or 64 weeks, or 65 weeks, or 66 weeks, or 67 weeks, or 68 weeks, or 69 weeks, or 70 weeks, or 71 weeks, or 72 weeks, or 73 weeks, or 74 weeks, or 75 weeks, or 76 weeks, or 77 weeks, or 78 weeks, or 79 weeks, or 80 weeks, or 81 weeks, or 82 weeks, or 83 weeks, or 84 weeks, or 85 weeks, or 86 weeks, or 87 weeks, or 88 weeks, or 89 weeks, or 90 weeks, or 91 weeks, or 92 weeks, or 93 weeks, or 94 weeks, or 95 weeks, or 96 weeks, or 97 weeks, or 98 weeks, or 99 weeks, or 100 weeks, or 101 weeks, or 102 weeks, or 103 weeks, or 104 weeks, 3 years, 4 years, 5 years, 6 years, 7 years, 8 years, 9 years, 10 years, 11 years, 12, years, 13 years, 14 years, 15 years, 16 years, 17 years, 18 years, 19 years, 20 years, or more.

### Oromucosal Spray

The oromucosal spray described herein comprises cannabidiol (CBD) and tetrahydrocannabinol (THC). In embodiments, THC is present as the trans isomer, the cis isomer, or a combination thereof. In embodiments, the oromucosal spray further comprises one or more additional cannabinoids, including, but not limited to cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof. In embodiments, the oromucosal spray further comprises one or more terpenes. In embodiments, the terpenes comprise one or more sesquiterpenes. Non-limiting examples of terpenes and sesquiterpenes include, but are not limited to, beta-farnesene, selina-3,7 (11)-diene, guaia-3,9-diene, trans-caryophyllene, alpha-caryophyllene, trans-nerolidol, myrcene, trans-phytol, squalene, alpha-tocopherol, or a combination thereof. In embodiments, the oromucosal spray further comprises one or more sterols, including but not limited to beta-sitosterol, beta-amyrin, campesterol, lupeol, or a combination thereof. Table 4 below provides the structure of certain cannabinoids, terpenes, and sterols along with their standard abbreviations. The table below is not exhaustive and merely details the cannabinoids and other potential components of the oromucosal spray composition which are identified in the present application for reference.

TABLE 4
Non-limiting Examples of Components of the Oromucosal Spray and Their
Abbreviations

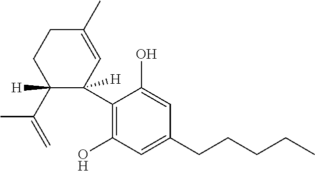 [SMILES: C=C(C)[C@@H]1CCC(C)=C[C@H]1c1c(O)cc(CCCCC)cc1O]

Cannabidiol (CBD)

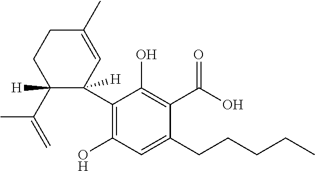 [SMILES: C=C(C)[C@@H]1CCC(C)=C[C@H]1c1c(O)cc(CCCCC)c(C(=O)O)c1O]

Cannabidiolic acid (CBDA)

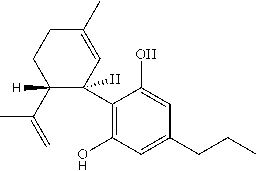 [SMILES: C=C(C)[C@@H]1CCC(C)=C[C@H]1c1c(O)cc(CCC)cc1O]

Cannabidivarin (CBDV)

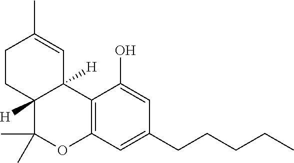 [SMILES: CCCCCc1cc(O)c2c(c1)OC(C)(C)[C@@H]1CCC(C)=C[C@@H]21]

trans-Tetrahydrocannabinol (THC)

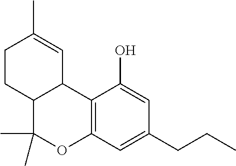 [SMILES: CCCc1cc(O)c2c(c1)OC(C)(C)C1CCC(C)=CC21]

Tetrahydrocannabivarin (THCV)

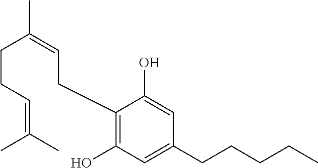 [SMILES: CCCCCc1cc(O)c(C/C=C(/C)CCC=C(C)C)c(O)c1]

Cannabigerol (CBG)

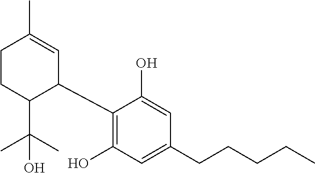 [SMILES: CCCCCc1cc(O)c(C2C=C(C)CCC2C(C)(C)O)c(O)c1]

OH-CBD (hydroxy cannabidiol)

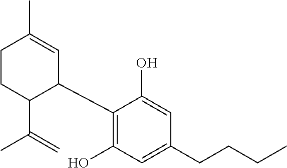 [SMILES: C=C(C)C1CCC(C)=CC1c1c(O)cc(CCCC)cc1O]

Butyl-cannabidiol (CBD-C4)

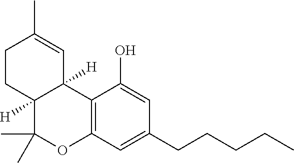 [SMILES: CCCCCc1cc(O)c2c(c1)OC(C)(C)[C@H]1CCC(C)=C[C@@H]21]

cis-THC (cis-Tetrahydrocannabinol)

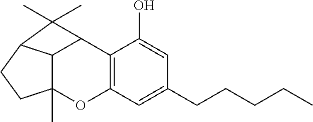 [SMILES: CCCCCc1cc(O)c2c(c1)OC1(C)CCC3C1C2C3(C)C]

CBL (Cannabicyclol)

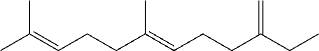

Beta-Farnesene

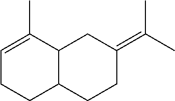

Selina-3,7(11)-diene

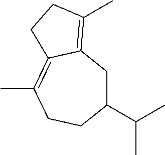

Guaia-3,9-diene

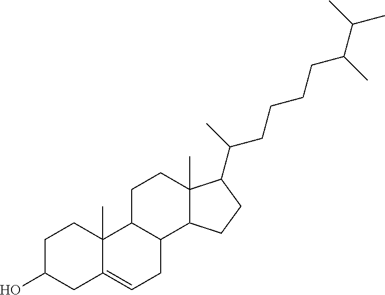 [SMILES: CC(C)C(C)CCCCC(C)C1CCC2C3CC=C4CC(O)CCC4(C)C3CCC12C]

Campesterol

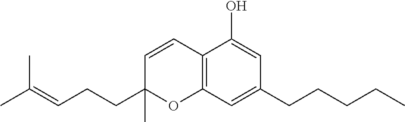 [SMILES: CCCCCc1cc(O)c2c(c1)OC(C)(CCC=C(C)C)C=C2]

Cannabichromene (CBC)

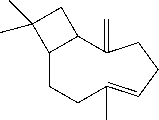

Trans-caryophyllene

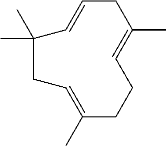

alpha-Caryophyllene

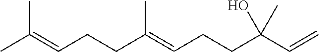 [SMILES: C=CC(C)(O)CC/C=C(\C)CCC=C(C)C]

Trans-nerolidol

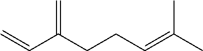

Myrcene

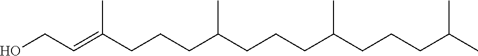 [SMILES: C/C(=C\CO)CCCC(C)CCCC(C)CCCC(C)C]

Trans-phytol

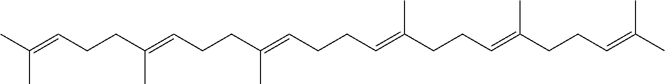 [SMILES: CC(C)=CCC/C(C)=C/CC/C(C)=C/CC/C=C(\C)CC/C=C(\C)CCC=C(C)C]

Squalene

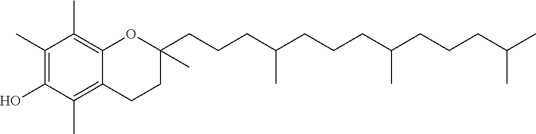 [SMILES: Cc1c(C)c2c(c(C)c1O)CCC(C)(CCCC(C)CCCC(C)CCCC(C)C)O2]

alpha-Tocopherol

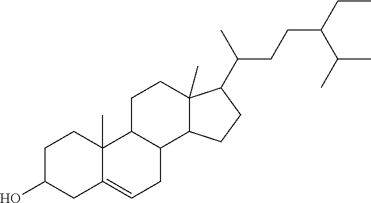 [SMILES: CCC(CCC(C)C1CCC2C3CC=C4CC(O)CCC4(C)C3CCC12C)C(C)C]

beta-Sitosterol

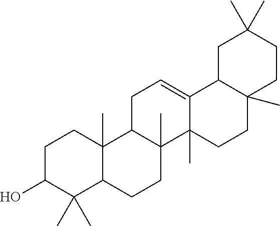

beta-Amyrin

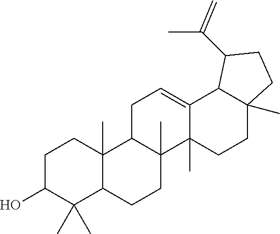

Lupeol

Without wishing to be bound by theory, the majority of cannabinoids (e.g., CBD and THC) are primarily metabolized in the liver and produce a plurality of metabolites. According to previous studies, CYP2C9 and CYP3A4 are indicated as the primary enzymes in the metabolism of CBD and THC in the liver. More than 100 THC metabolites are currently identified, which are mostly mono-hydroxylated compounds such as 11-OH-THC and 11-COOH-THC. Examples of CBD metabolites include 7-OH-CBD, and 7-COOH-CBD. Among these metabolites, 7-OH-CBD and 11-OH-THC are pharmacologically active intermediate metabolites of CBD and THC and are at least equally potent as compared to the parent molecules in producing their pharmacological effects in certain disorders (e.g., seizure, psychiatric disorders, etc.). All metabolites of THC and CBD are contemplated to be included within this disclosure. Table 5 shows the structures certain metabolites of CBD and THC.

TABLE 5
Structures of 11-COOH-THC, 11-OH-THC, 7-OH-CBD, and
7-COOH-CBD

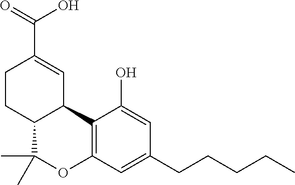 [SMILES: CCCCCc1cc(O)c2c(c1)OC(C)(C)[C@@H]1CCC(C(=O)O)=C[C@@H]21]

11-COOH-THC

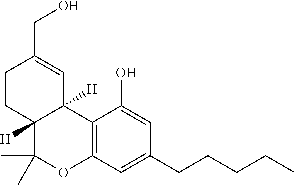 [SMILES: CCCCCc1cc(O)c2c(c1)OC(C)(C)[C@@H]1CCC(CO)=C[C@@H]21]

11-OH-THC

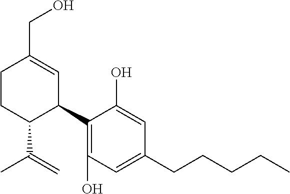 [SMILES: C=C(C)[C@@H]1CCC(CO)=C[C@H]1c1c(O)cc(CCCCC)cc1O]

7-OH-CBD

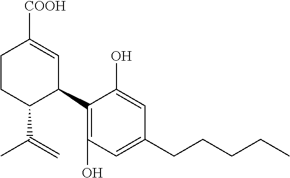 [SMILES: C=C(C)[C@@H]1CCC(C(=O)O)=C[C@H]1c1c(O)cc(CCCCC)cc1O]

7-COOH-CBD

The cannabinoids, terpenes, and sterols described herein may be prepared synthetically or naturally from Cannabis. Cannabis is a genus of flowering plants in the family Cannabaceae, comprising the species Cannabis sativa, Cannabis indica, and Cannabis ruderalis. In embodiments, CBD and THC are prepared synthetically. In embodiments, both CBD and THC are extracted from Cannabis. When CBD and THC are extracted from plants, the drug substance may be referred to as a botanical drug substance. In embodiments, CBD and THC are present in the oromuscal spray as purified extracts. When CBD and THC are extracted and used in the oromucosal spray, CBD and THC may be the predominant cannabinoids in the oromucosal spray, but other cannabinoids or components (e.g., terpenes, sterols, etc.) may also exist in the oromucosal spray. Accordingly, in embodiments, the oromucosal spray described herein further comprises (in addition to CBD and THC), CBDA, CBDV, CBN, CBC, mono-methylated CBG (CBG MME), CBD-C1, CBD-C4, THCV, CBG, OH-CBD, CBL, DHC, and/or various terpenes and sterols described herein (e.g., alpha-bergmatone, alpha-bisbolol, beta-farnesene, selina-3,7 (11)-diene, guaia-3,9-diene, trans-caryophyllene, alpha-caryophyllene, trans-nerolidol, myrcene, trans-phytol, squalene, alpha-tocopherol, beta-sitosterol, beta-amyrin, campesterol, lupeol, or combinations thereof).

In embodiments, the oromucosal spray comprises CBD. In embodiments, CBD is extracted from a Cannabis. In embodiments, CBD is extracted from Cannabis and purified. In embodiments, the purified CBD extract comprises from 50% w/w to at least 99% w/w CBD based on the total weight of the extract. In embodiments, the CBD extract can comprise about 50% w/w, about 55% w/w, about 60% w/w, about 65% w/w, about 70% w/w, about 75% w/w, about 80% w/w, about 85% w/w, about 90% w/w, 95% w/w, about 96% w/w, about 97% w/w, about 98% w/w, about 99% w/w, or about 100% w/w CBD based on the total weight of cannabinoids in the extract, including all value sand ranges therein. In embodiments, the CBD extract comprises at least 60% w/w CBD based on the total weight of the extract. In embodiments, the CBD extract comprises 98-99% w/w CBD based on the total weight of the extract. In embodiments, the CBD extract further comprises OH-CBD, CBDV, CBG, THC, CBC, CBG MME, Myrcene, Trans-Caryophellene, Alpha-Caryophyllene, Trans-Nerolidol, Trans-Phytol, Squalene, Alpha-Tocopherol, Beta-Sitosterol, or Beta-Amyrin, or combinations thereof. In embodiments, the purified CBD extract may be combined with THC to form the oromucosal spray described herein. In embodiments, THC be prepared either synthetic or purified from Cannabis, and combined with the CBD. Methods for CBD synthesis and extraction are described in the following patent documents which are incorporated by reference in their entirety herein: U.S. Publication No. 2019/0231833 A1, (published Aug. 1, 2019), International Publication No. 2019/020738 (published Jan. 31, 2019), International Publication No. 2004/016277 A1 (published Feb. 26, 2004), U.S. Publication No. 2019/0160393 A1 (published May 30, 2019), and International Publication No. 2004/026802 (published Jan. 4, 2004).

In embodiments, the amount of CBD in the oromucosal spray may range from about 1 mg/mL to about 100 mg/mL, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein. In some embodiment, the spray comprises about 20 mg/mL to about 30 mg/mL of CBD (e.g., about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, about 25 mg/mL, about 25.5 mg/mL, about 26 mg/mL, about 26.5 mg/mL, about 27 mg/mL, about 27.5 mg/mL, about 28 mg/mL, about 28.5 mg/mL, about 29 mg/mL, about 29.5 mg/mL, about 30 mg/mL, including all values and ranges therein). In embodiments, the spray comprises 22.5 mg/mL to about 27.5 mg/mL of CBD. In embodiments, the spray comprises 23.7 mg/mL to about 26.5 mg/mL of CBD.

In embodiments, the oromucosal spray comprises THC. In embodiments, the THC extract comprises from 50% w/w to 100% w/w THC based on the total weight of the extract. In embodiments, the THC extract can comprise about 50% w/w, about 55% w/w, about 60% w/w, about 65% w/w, about 70% w/w, about 75% w/w, about 80% w/w, about 85% w/w, about 90% w/w, 95% w/w, about 96% w/w, about 97% w/w, about 98% w/w, about 99% w/w THC based on the total weight of the extract, including all values and ranges therein. In embodiments, the THC extract comprises at least 64% w/w THC based on the total weight of the extract. In embodiments, the THC extract comprises 98-99% w/w THC based on the total weight of cannabinoids in the extract. In some embodiments, the THC extract further comprises THCV, CBD, CBG, CBN, DHC, CBC, Myrcene, Trans-Caryophellene, Alpha-Bergamotene, Beta-Farnesene, Alpha-Caryophyllene, Guaia-3,9-diene, Selina-3,7 (11)-diene, Trans-Nerolidol, Alpha-Bisabolol, Trans-Phytol, Beta-Sitosterol, Beta-Amyrin, Alpha-Amyrin, or Lupeol, or combinations thereof. Methods for THC extraction are described in U.S. Publication No. 2005/0266108 (published Dec. 1, 2005) which is herein incorporated by reference in its entirety.

In embodiments, the amount of THC in the spray composition can range from about 1 mg/mL to about 100 mg/mL, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein. In embodiments, the spray comprises about 20 mg/mL to about 30 mg/mL of THC (e.g., about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, about 25 mg/mL, about 25.5 mg/mL, about 26 mg/mL, about 26.5 mg/mL, about 27 mg/mL, about 27.5 mg/mL, about 28 mg/mL, about 28.5 mg/mL, about 29 mg/mL, about 29.5 mg/mL, about 30 mg/mL, including all values and ranges therein). In embodiments, the spray comprises 22.5 mg/mL to about 27.5 mg/mL of THC. In embodiments, the spray comprises 24.3 mg/mL to about 28.4 mg/mL of THC.

In embodiments, the spray comprises about 1 mg/mL to about 100 mg/mL of THC (for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein), and 1 mg/mL to about 100 mg/mL of CBD, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein.

In embodiments, the oromucosal spray comprises about 20-30 mg/mL THC and about 20-30 mg/mL CBD. In embodiments, the oromucosal spray comprises about 22.5 mg/mL to about 27.5 mg/mL of THC (for example, 22.5 mg/mL, 22.6 mg/mL, 22.7 mg/mL, 22.8 mg/mL, 22.9 mg/mL, 23.0 mg/mL, 23.1 mg/mL, 23.2 mg/mL, 23.3 mg/mL, 23.4 mg/mL, 23.5 mg/mL, 23.6 mg/mL, 23.7 mg/mL, 23.8 mg/mL, 23.9 mg/mL, 24.0 mg/mL, 24.1 mg/mL, 24.2 mg/mL, 24.3 mg/mL, 24.4 mg/mL, 24.5 mg/mL, 24.6 mg/mL, 24.7 mg/mL, 24.8 mg/mL, 24.9 mg/mL, 25.0 mg/mL, 25.1 mg/mL, 25.2 mg/mL, 25.3 mg/mL, 25.4 mg/mL, 25.5 mg/mL, 25.6 mg/mL, 25.7 mg/mL, 25.8 mg/mL, 25.9 mg/mL, 26.0 mg/mL, 26.1 mg/mL, 26.2 mg/mL, 26.3 mg/mL, 26.4 mg/mL, 26.5 mg/mL, 26.6 mg/mL, 26.7 mg/mL, 26.8 mg/mL, 26.9 mg/mL, 27.0 mg/mL, 27.1 mg/mL, 27.2 mg/mL, 27.3 mg/mL, 27.4 mg/mL, 27.5 mg/mL, including all values and ranges therein), and about 22.5 mg/mL to about 27.5 mg/mL of CBD (for example, 22.5 mg/mL, 22.6 mg/mL, 22.7 mg/mL, 22.8 mg/mL, 22.9 mg/mL, 23.0 mg/mL, 23.1 mg/mL, 23.2 mg/mL, 23.3 mg/mL, 23.4 mg/mL, 23.5 mg/mL, 23.6 mg/mL, 23.7 mg/mL, 23.8 mg/mL, 23.9 mg/mL, 24.0 mg/mL, 24.1 mg/mL, 24.2 mg/mL, 24.3 mg/mL, 24.4 mg/mL, 24.5 mg/mL, 24.6 mg/mL, 24.7 mg/mL, 24.8 mg/mL, 24.9 mg/mL, 25.0 mg/mL, 25.1 mg/mL, 25.2 mg/mL, 25.3 mg/mL, 25.4 mg/mL, 25.5 mg/mL, 25.6 mg/mL, 25.7 mg/mL, 25.8 mg/mL, 25.9 mg/mL, 26.0 mg/mL, 26.1 mg/mL, 26.2 mg/mL, 26.3 mg/mL, 26.4 mg/mL, 26.5 mg/mL, 26.6 mg/mL, 26.7 mg/mL, 26.8 mg/mL, 26.9 mg/mL, 27.0 mg/mL, 27.1 mg/mL, 27.2 mg/mL, 27.3 mg/mL, 27.4 mg/mL, 27.5 mg/mL, including all values and ranges therein). In embodiments, the spray comprises 22.5 mg/mL to about 27.5 mg/mL of THC, and 22.5 mg/mL to about 27.5 mg/mL of CBD. In embodiments, the spray comprises 24.3 mg/mL to about 28.4 mg/mL of THC, and about 23.7 mg/mL to about 26.5 mg/mL of CBD. In embodiments, the oromucosal spray comprises about 27 mg/mL of THC and 25 mg/mL of CBD.

In embodiments, the THC and CBD are present in a ratio by weight of from 0.5:1.5 to 1.5:0.5. In embodiments, the THC and CBD are present in a ratio by weight of from 0.7:1.3 to 1.3:0.7. In embodiments, the THC and CBD are present in a ratio by weight of from 0.9:1.1 to 1.1:0.9. In embodiments, the THC and CBD are present in a ratio by weight of 1.1:0.9.

In embodiments, the oromucosal spray releases or delivers about 50 μL to about 200 μL (e.g., about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, or about 200 μL, including all values and ranges therein) per spray. In embodiments, the oromucosal spray releases or delivers about 100 μL per spray. In embodiments, the oromucosal spray releases or delivers about 2 to about 3 mg of THC per spray (for example, 2.00 mg, 2.01 mg, 2.02 mg, 2.03 mg, 2.04 mg, 2.05 mg, 2.06 mg, 2.07 mg, 2.08 mg, 2.09 mg, 2.10 mg, 2.11 mg, 2.12 mg, 2.13 mg, 2.14 mg, 2.15 mg, 2.16 mg, 2.17 mg, 2.18 mg, 2.19 mg, 2.20 mg, 2.21 mg, 2.22 mg, 2.23 mg, 2.24 mg, 2.25 mg, 2.26 mg, 2.27 mg, 2.28 mg, 2.29 mg, 2.30 mg, 2.31 mg, 2.32 mg, 2.33 mg, 2.34 mg, 2.35 mg, 2.36 mg, 2.37 mg, 2.38 mg, 2.39 mg, 2.40 mg, 2.41 mg, 2.42 mg, 2.43 mg, 2.44 mg, 2.45 mg, 2.46 mg, 2.47 mg, 2.48 mg, 2.49 mg, 2.50 mg, 2.51 mg, 2.52 mg, 2.53 mg, 2.54 mg, 2.55 mg, 2.56 mg, 2.57 mg, 2.58 mg, 2.59 mg, 2.60 mg, 2.61 mg, 2.62 mg, 2.63 mg, 2.64 mg, 2.65 mg, 2.66 mg, 2.67 mg, 2.68 mg, 2.69 mg, 2.70 mg, 2.71 mg, 2.72 mg, 2.73 mg, 2.74 mg, 2.75 mg, 2.76 mg, 2.77 mg, 2.78 mg, 2.79 mg, 2.80 mg, 2.81 mg, 2.82 mg, 2.83 mg, 2.84 mg, 2.85 mg, 2.86 mg, 2.87 mg, 2.88 mg, 2.89 mg, 2.90 mg, 2.91 mg, 2.92 mg, 2.93 mg, 2.94 mg, 2.95 mg, 2.96 mg, 2.97 mg, 2.98 mg, 2.99 mg, 3.00 mg, including all values and ranges therein), and about 2 to about 3 mg of CBD per spray (for example, 2.25 mg, 2.26 mg, 2.27 mg, 2.28 mg, 2.29 mg, 2.30 mg, 2.31 mg, 2.32 mg, 2.33 mg, 2.34 mg, 2.35 mg, 2.36 mg, 2.37 mg, 2.38 mg, 2.39 mg, 2.40 mg, 2.41 mg, 2.42 mg, 2.43 mg, 2.44 mg, 2.45 mg, 2.46 mg, 2.47 mg, 2.48 mg, 2.49 mg, 2.50 mg, 2.51 mg, 2.52 mg, 2.53 mg, 2.54 mg, 2.55 mg, 2.56 mg, 2.57 mg, 2.58 mg, 2.59 mg, 2.60 mg, 2.61 mg, 2.62 mg, 2.63 mg, 2.64 mg, 2.65 mg, 2.66 mg, 2.67 mg, 2.68 mg, 2.69 mg, 2.70 mg, 2.71 mg, 2.72 mg, 2.73 mg, 2.74 mg, 2.75 mg, 2.76 mg, 2.77 mg, 2.78 mg, 2.79 mg, 2.80 mg, 2.81 mg, 2.82 mg, 2.83 mg, 2.84 mg, 2.85 mg, 2.86 mg, 2.87 mg, 2.88 mg, 2.89 mg, 2.90 mg, 2.91 mg, 2.92 mg, 2.93 mg, 2.94 mg, 2.95 mg, 2.96 mg, 2.97 mg, 2.98 mg, 2.99 mg, 3.00 mg, including all values and ranges therein). In embodiments, the oromucosal spray releases or delivers about 2.25 mg to about 2.75 mg of THC (e.g., about 2.25 mg, about 2.3 mg, about 2.35 mg, about 2.4 mg, about 2.45 mg, about 2.5 mg, about 2.55 mg, about 2.65 mg, about 2.7 mg, or about 2.75 mg) and about 2.25 to about 2.75 mg of CBD (e.g., about 2.25 mg, about 2.3 mg, about 2.35 mg, about 2.4 mg, about 2.45 mg, about 2.5 mg, about 2.55 mg, about 2.65 mg, about 2.7 mg, or about 2.75 mg). In embodiments, the oromucosal spray releases or delivers about 2.7 mg of THC and about 2.5 mg of CBD.

The oromucosal spray described herein may contain any suitable pharmaceutically acceptable excipient. In embodiments, the oromucosal spray comprises a pharmaceutically acceptable solvent suitable to dissolve cannabinoids (e.g., THC and CBD). In embodiments, the solvent comprises a C1-C4 alcohol. In embodiments, the solvent comprises ethanol. The solvent and co-solvent may be present in an appropriate amount to solubilize the cannabinoids and allow delivery of the cannabinoids to the oral cavity via a spray. In embodiments, the solvent is present in an amount ranging from about 50%-98% w/w based on the total weight of the oromucosal spray (e.g., about 50%, about 55%, about 60%, about 65%, about 70%, about 75%, about 80%, about 85%, about 90%, about 91%, about 92%, about 93%, about 94%, about 95%, about 96%, about 97%, or about 98% v/v, based on the total weight of the oromucosal spray). In embodiments, the solvent is present in amount of at least about 65% w/w. In embodiments, the solvent is present in amount of at least about 70% w/w. In embodiments, the solvent is present in amount of at least about 75% w/w. In embodiments, the solvent is present in amount of at least about 80% w/w. In embodiments, the solvent is present in amount of at least about 85% w/w. In embodiments, the solvent is present in amount ranging from 80% w/w to about 98% w/w of the formulation.

In embodiments, the oromucosal spray further a pharmaceutically acceptable co-solvent. In embodiments, co-solvent is a glycol, sugar alcohol, carbonate ester or chlorinated hydrocarbons. In embodiments, co-solvent is a glycol. In embodiments, the glycerol is propylene glycol or glycerol. In embodiments, the glycerol is propylene glycol. In embodiments, the co-solvent is a carbonate ester. In embodiments, the carbonate ester is propylene carbonate. In embodiments, the co-solvent is propylene glycol. In embodiments, the solvent is ethanol, and the co-solvent is propylene glycol.

In embodiments, the solvent and co-solvent are present in a weight ratio ranging from 60/40 to 40/60 (e.g., 60/40, 59/41, 58/42, 57/43, 56/44, 55/45, 54/46, 53/47, 52/48, 51/49, 50/50, 49/51, 48/52, 47/53, 46/54, 45/55, 44/56, 43/57, 42/58, 41/59, 40/60, including all subranges therein). In embodiments, the solvent and co-solvent are present in a weight ratio in the range 55/45 to 45/55. In embodiments, the solvent and co-solvent are present in a weight ratio of about 50/50.

In embodiments, the solvent is ethanol and the co-solvent is propylene glycol. In embodiments, ethanol/propylene glycol are present in weight ratio ranging from 55/45 to 45/55. In embodiments, ethanol/propylene glycol are present in a weight ratio of about 50/50.

The solvent and co-solvent may be present in an appropriate amount to solubilize the cannabinoids and allow delivery of the cannabinoids to the oral cavity via a spray. In embodiments, the solvent and co-solvent are present in an amount ranging from about 50%-98% w/w based on the total weight of the oromucosal spray (e.g., about 50%, about 55%, about 60%, about 65%, about 70%, about 75%, about 80%, about 85%, about 90%, about 91%, about 92%, about 93%, about 94%, about 95%, about 96%, about 97%, or about 98% v/v, based on the total weight of the oromucosal spray). In embodiments, the solvent and co-solvent are present in amount of at least about 65% w/w. In embodiments, the solvent and co-solvent are present in amount of at least about 70% w/w. In embodiments, the solvent and co-solvent are present in amount of at least about 75% w/w. In embodiments, the solvent and co-solvent are present in amount of at least about 80% w/w. In embodiments, the solvent and co-solvent are present in amount of at least about 85% w/w. In embodiments, the solvent and co-solvent are present in amount ranging from 80% w/w to about 98% w/w of the formulation.

In embodiments, the oromucosal spray comprises flavoring. In embodiments, the flavoring is strawberry, cherry, peppermint, orange, grape, or other flavors. In embodiments, the flavoring is peppermint oil. In embodiments, the flavoring is present in a suitable amount such that the formulation is palatable. In embodiments, the flavoring is present in a suitable amount to mask the taste of the cannabinoids. In embodiments, the flavoring is present in an amount up to 0.1% v/v based on the total volume of the oromucosal spray, e.g., 0.01, 0.02, 0.03, 0.04, 0.05, 0.06, 0.07, 0.08, 0.09, or 0.1% v/v, including all values and ranges therein.

Non-limiting examples of an oromucosal spray comprising CBD and THC of the present disclosure are described in U.S. Pat. No. 8,603,515 (Published May 2, 2013), which is incorporated by reference its entirety herein.

In embodiments, the oromucosal spray comprises THC and CBD (e.g., in the amounts described herein), and one or more of OH-CBD, CBDV, THCV, CBG, CBN, DHC, CBC, mono-methylated CBG (CBG MME), or any combination thereof. In embodiments, the oromucosal spray comprises about 20-30 mg/mL THC (for example, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, including all values therein), 20-30 mg/mL CBD (for example, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, including all values therein), 0.1-0.8 mg/mL OH-CBD (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, including all values therein), 0.1-0.6 mg/mL CBDV (for example, 0.1 mg/mL, 0.2 mg/mL, 0.3 mg/mL, 0.4 mg/mL, 0.5 mg/mL, 0.6 mg/mL, including all values therein), 0.1-0.8 mg/mL THCV (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, including all values therein), 0.2-3.2 mg/mL CBG (for example, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, 3.1 mg/mL, 3.2 mg/mL, including all values therein), 0.1-1.8 mg/mL CBN (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 0.1 mg/mL, 1.2 mg/mL, 1.3 mg/mL, 1.4 mg/mL, 1.5 mg/mL, 1.6 mg/mL, 1.7 mg/mL, 1.8 mg/mL, including all values therein), 0.05-0.3 mg/mL DHC (for example, 0.05 mg/mL, 0.1 mg/mL, 0.2 mg/mL, 0.3 mg/mL, including all values therein), 1.0-5.0 mg/mL CBC (for example, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, including all values therein), and 0.05-0.4 mg/mL CBG MME (for example, 0.05 mg/mL, 0.1 mg/mL, 0.2 mg/mL, 0.3 mg/mL, 0.4 mg/mL, including all values therein). In embodiments, the oromucosal spray comprises 24.3-28.4 mg/mL THC, 23.7-26.5 mg/mL CBD, 0.1-0.4 mg/mL OH-CBD, 0.1-0.3 mg/mL CBDV, 0.1-0.4 mg/mL THCV, 0.2-1.6 mg/mL CBG, 0.1-0.9 mg/mL CBN, 0.05-0.3 mg/mL DHC, 1.0-2.8 mg/mL CBC, and 0.05-0.4 mg/mL CBG MME.

As discussed herein, in some embodiments, the oromucosal spray further comprises (in addition to CBD and THC, and optionally one or more additional cannabinoids described herein) one or more terpenes. In embodiments, the total terpene content in the oromucosal spray ranges from 1.1 mg/mL to 8.3 mg/mL (e.g., 1.1 mg/mL mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.3 mg/mL, including all values therein). In embodiments, the oromucosal spray further comprises 0.05-1.6 mg/mL Myrcene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, including all values therein), 0.1-5.0 mg/mL Trans-Caryophyllene (for example, 0.1 mg/mL, 0.2 mg/mL, 0.3 mg/mL, 0.4 mg/mL, 0.5 mg/mL, 1 mg/mL, 1.5 mg/mL, 2 mg/mL, 2.5 mg/mL, 3 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, including all values therein), 0.05-1.0 mg/mL Alpha-Bergamotene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-1.0 mg/mL Beta-Farnesene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-2.0 mg/mL Alpha-Caryophyllene (for example, 0.05 mg/mL, 0.1 mg/mL, 0.2 mg/mL, 0.3 mg/mL, 0.4 mg/mL, 0.5 mg/mL, 1.0 mg/mL, 1.5 mg/mL, 2.0 mg/mL, including all values therein), 0.05-1.0 mg/mL Guaia-3,9-diene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-1.0 mg/mL Selina-3,7 (11)-diene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.1-1.0 mg/mL Trans-Nerolidol (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-0.5 mg/mL Alpha-Bisabolol (for example, 0.05 mg/mL, 0.1 mg/mL, 0.2 mg/mL, 0.3 mg/mL, 0.4 mg/mL, 0.5 mg/mL, including all values therein), 0.1-1.5 mg/mL Trans-Phytol (for example, 0.1 mg/mL, 0.2 mg/mL, 0.3 mg/mL, 0.4 mg/mL, 0.5 mg/mL, 0.6 mg/mL, 0.7 mg/mL, including all values therein). In embodiments, the oromucosal spray further comprises no more than 0.8 mg/mL Myrcene, 0.1-2.8 mg/mL Trans-Caryophyllene, not more than 0.3 mg/mL Alpha-Bergamotene, not more than 0.5 mg/mL Beta-Farnesene, 0.1-1.1 mg/mL Alpha-Caryophyllene, not more than 0.5 mg/mL Guaia-3,9-diene, nor more than 0.6 mg/mL Selina-3,7 (11)-diene, 0.1-0.4 mg/mL Trans-Nerolidol, not more than 0.3 mg/mL Alpha-Bisabolol, 0.1-0.7 mg/mL Trans-Phytol.

In embodiments, the oromucosal spray comprises components listed in Table 6.

TABLE 6
Example composition

| Component | Concentration | Component | Concentration |
|---|---|---|---|
| THC | 24.3-28.4 mg/mL | Myrcene | NMT 0.8 mg/mL |
| CBD | 23.7-26.5 mg/mL | Trans-Caryophyllene | 0.1-2.8 mg/mL |
| OH-CBD | 0.1-0.4 mg/mL | Alpha-Bergamotene | NMT 0.3 mg/mL |
| CBDV | 0.1-0.3 mg/mL | Beta-Farnesene | NMT 0.5 mg/mL |
| THCV | 0.1-0.4 mg/mL | Alpha-Caryophyllene | 0.1-1.1 mg/mL |
| CBG | 0.2-1.6 mg/mL | Guaia-3,9-diene | NMT 0.5 mg/mL |
| CBN | 0.1-0.9 mg/mL | Selina-3,7(11)-diene | NMT 0.6 mg/mL |
| DHC | NMT 0.3 mg/mL | Trans-Nerolidol | 0.1-0.4 mg/mL |
| CBC | 1.0-2.8 mg/mL | Alpha-Bisabolol | NMT 0.3 mg/mL |
| CBG MME | NMT 0.4 mg/mL | Trans-Phytol | 0.1-0.7 mg/mL |

NMT: Not More Than

### Clinical Outcomes

The methods of this disclosure are used to treat patients with varying degrees of renal impairment who are suffering from long-term pain associated with cancer, or MS-associated spasticity or neuropathic pain. In embodiments, the methods comprise treating MS-associated spasticity. In embodiments, efficacy of treatment of MS-associated spasticity is determined by measuring spasticity numerical rating scale (NRS) score during the administration period. For instance, the spasticity is scored on an 11-point NRS from 0 (no spasticity) to 10 (worst imaginable spasticity), and patients indicate the average level of their spasticity related symptoms over the last 24 hours. Further details on spasticity NRS are provided in Farrar et al. Clinical Therapeutics 2008, 30 (5), 974-985, the contents of which are herein incorporated by reference in its entirety. In embodiments, the patient has at least moderate spasticity and reports NRS score of 4 or more prior to treatment. In embodiments, the NRS score identified by the patient prior to treatment is 4, 4.5, 5, 5.5, 6, 6.5, 7, 7.5, 8, 8.5, 9, 9.5, or 10. In embodiments, secondary evaluation methods are utilized to determine the clinical efficacy of the oromucosal spray, and examples of evaluation include modified Ashworth score for spasticity, spasm frequency, sleep disruption by spasticity in NRS, timed 10-meter walk (in seconds), motricity index (arm and leg), and Barthel activities of daily living.

In embodiments, the NRS score of the patient during the administration period is less than the NRS score of the patient prior to treatment. In embodiments, the NRS score of the patient during the administration period is at least about 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the NRS score of the patient prior to treatment. In embodiments, the NRS score of the patient during the administration period is at least about 1 point less (e.g., 1, 1.5, 2, 2.5, 3, 3.5, 4, 4.5, 5, 5.5, 6, 6.5, 7, 7.5, 8, 8.5, 9, 9.5, or 10) than the NRS score of the patient prior to treatment.

In embodiments, the methods comprise reducing or treating neuropathic pain in a patient with multiple sclerosis (MS). In embodiments, efficacy of the reduction in neuropathic pain according to the methods of the disclosure is determined by measuring the changes in the pain severity score on the BS-11, 11-box numerical rating scale (NRS) system during the administration period or subsequent to the administration period. For instance, the neuropathic pain is scored on an 11-point NRS from 0 to 10 where 0 indicates “no pain” and 10 indicates “maximum pain”. The patient is instructed to identify one number between 0 and 10, which is best representative of their pain intensity. Further details on 11-point NRS are provided in Sharma et al. Health Qual Life Outcomes 2017, 15:236, the contents of which are herein incorporated by reference in its entirety. In embodiments, the patient has at least moderate or severe pain and identifies the pain NRS score of 4 or more prior to the administration period. In embodiments, the pain NRS score identified by the patient prior to the administration period is 4, 4.5, 5, 5.5, 6, 6.5, 7, 7.5, 8, 8.5, 9, 9.5, or 10.

In embodiments, the pain severity NRS score of the patient during the administration period is less than the pain severity NRS score of the patient prior to the administration period. In embodiments, the pain severity NRS score of the patient during the administration period is at least about 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the pain severity NRS score of the patient prior to the administration period. In embodiments, the NRS score of the patient during the administration period is at least about 1 point less (e.g., 1, 1.5, 2, 2.5, 3, 3.5, 4, 4.5, 5, 5.5, 6, 6.5, 7, 7.5, 8, 8.5, or 9) than the NRS score of the patient prior to treatment.

In embodiments, the methods comprise reducing or treating pain in a patient with advanced cancer. In embodiments, as described above, efficacy of the reduction in pain according to the methods of the disclosure is determined by measuring the changes in the mean BS-11, 11-box numerical rating scale (NRS) score during the administration period or subsequent to the administration period. For instance, 11-point NRS from 0 to 10 where 0 indicates “no pain” and 10 indicates “maximum pain”. The patient is instructed to identify one number between 0 and 10, which is best representative of their pain intensity. In embodiments, the patient reports the pain NRS score of about 4 or more or more prior to the administration period. In embodiments, the patient reports moderate to severe pain despite already taking an opioid for chronic pain. In embodiments, the pain NRS score identified by the patient prior to the administration period is 4, 4.5, 5, 5.5, 6, 6.5, 7, 7.5, 8, 8.5, 9, 9.5, or 10.

In embodiments, the pain severity NRS score of the patient with advanced cancer during the administration period is less than the pain severity NRS score of the patient prior to treatment. In embodiments, the pain severity NRS score of the patient during the administration period is at least about 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the pain severity NRS score of the patient prior to treatment. In embodiments, the NRS score of the patient during the administration period is at least about 1 point less (e.g., 1, 1.5, 2, 2.5, 3, 3.5, 4, 4.5, 5, 5.5, 6, 6.5, 7, 7.5, 8, 8.5, 9, 9.5, or 10) than the NRS score of the patient prior to treatment.

### EXAMPLES

### Example 1. A Phase 1, Open-Label, Single-Dose Trial to Investigate the Pharmacokinetics of Oromucosal Spray Described Herein in Participants with Severe Renal Impairment or End Stage Renal Disease

Rationale: To date, no studies of administering the disclosed oromucosal spray in patients with varying degrees of renal impairment have been conducted. The present trial investigated the effects of the oromucosal spray in subjects with severe renal impairment. The principal aim of this trial was to assess the pharmacokinetic (PK) parameters following oromucosal administration in patients with severe renal impairment and individually matched healthy participants (i.e., otherwise identical patients with normal renal function).

Primary Objectives: Evaluated and compared the PK parameters of the oromucosal spray in participants with severe renal impairment or End-Stage Renal Disease (ESRD) to the PK profile in matched participants with normal renal function.

Primary Endpoints: The PK parameters, derived from the plasma concentration-time (Cₘₐₓ) and area under the concentration-time curve calculated to the last concentration at time t (AUC₀₋ₜ), or 48 hours (AUC₀₋₄₈), for THC, CBD, and metabolites of THC and CBD (e.g., 11-OH-THC and 7-COOH-CBD).

Secondary Objectives: 1) Additional plasma and urinary PK parameters of THC and CBD and their major metabolites following an oromucosal dose of nabiximols in participants with severe renal impairment or ESRD not requiring dialysis, and in matched participants with normal renal function. 2) Safety and tolerability of 4 sprays (10.8 mg THC and 10 mg CBD) in participants with severe renal impairment or ESRD but not requiring dialysis.

Secondary Endpoints: 1) pharmacokinetic endpoints include half-life (t½), time to maximum plasma concentration (tₘₐₓ), t½ apparent, apparent clearance of drug from plasma (CL/F), renal clearance (CLR), apparent volume of distribution (Vz/F), and/or fraction of the systemically available drug excreted into the urine (fe) of THC, CBD, and corresponding metabolites; 2) safety includes: adverse events (AEs), physical and oral examination, 12-lead electrocardiogram (ECG), clinical laboratory parameters (biochemistry, hematology, and urinalysis) and vital signs (blood pressure and pulse rate), derived from a 4 sprays in renally impaired or normal participants. The PK endpoints were measured 48 hours postdose, and safety endpoints were collected 14 days after dosing.

Design: This clinical study evaluated the effect of an oromucosal dosing (4 sprays) in participants with severe renal impairment or ESRD with an estimated glomerular filtration rate [eGFR]<30 mL/min/1.73 m², and in matched healthy participants with normal renal function having an estimated glomerular filtration rate [eGFR]≥90 mL/min/1.73 m². The 4 sprays administered a total of 10.8 mg THC and 10 mg CBD. The duration of the trial was approximately 45 days, which included a screening period (up to 4 weeks), a treatment period (48 hours) and a safety follow-up period (2 weeks). Screening was performed within 28 to 2 days before dosing, and the assessments included measurement of serum creatinine to assess the eGFR as determined by the modification of diet in renal disease formula (MDRD) to verify initial participant eligibility for trial participation. Participants entered the screening period (Day −28 to −1). From Day −1, screened participants who continued to meet eligibility criteria were admitted to the clinical research facility and placed under observation continually from dosing until 48 hours postdose. At Day −1, a blood sample was collected for assessment of eGFR to re-confirm eligibility for trial participation and to determine which group the participant could be placed in (either renally impaired group or normal control group). On Day 1, participants received 4 sprays of the oromucosal spray.

Formulation Mode of Administration, Dose, and Regimen: The oromucosal spray was formulated as a mixture of THC and CBD extracts derived from Cannabis sativa L. The formulation contains THC and CBD as the most abundant cannabinoid constituents and also contains minor constituents including related cannabinoids and non-cannabinoid plant components, such as terpenes, sterols, and triglycerides. The spray contains 27 mg/mL of THC and 25 mg/mL of CBD dissolved in the excipients (e.g., ethanol and propylene glycol). Each spray delivers 100 μL containing 2.7 mg of THC and 2.5 mg of CBD.

Procedures: PK samplings were taken as close as to the nominal time point. Participants fasted overnight for at least 10 hours prior to the administration and for at least 4 hours thereafter. Standardized meals and beverages were provided at approximately 4 hours and 9 hours after dosing. With the exception of the water administered at the time of dosing, water was not permitted from 1 hour before until 1 hour after dosing. Participants did not consume any food and beverages not provided during the inpatient period. Cannabis or use of anticonvulsants, antidepressants, antipsychotic and anxiolytic medications were not allowed throughout the trial. Alcohol, nicotine products, and consuming food or beverages containing methylxanthines (caffeinated food or beverages) were prohibited for 24 hours prior to each visit and throughout the inpatient period.

The following assessments were performed during screening: demographics, medical history, physical examination, vital signs, body weight, height, 12-lead ECG, adverse events (AEs), previous and concomitant medications history were recorded. The investigator or delegate completed the C-SSRS. Clinical laboratory samples were used in screening including chemistry, hematology, serology, urine drug screen, alcohol test, and pregnancy test.

Inclusion Criteria: Male and female subjects ≥18 and ≤60 years of age at the time of informed consent. Participants had a body weight of at least 50.0 kg and has a body mass index (BMI) between 18.5 and 30.0 kg/m², inclusive, at screening. Normal control group participants were individually matched to renally impaired participants with respect to age (within the decile or ±5 years, whichever is less), sex, and BMI (±10% BMI).

For the renally impaired participants group, participants who had severe renal impairment or ESRD (with eGFR<30 mL/min/1.73 m²) and not in need of dialysis were considered. The onset of renal impairment of participant from at least 3 months prior to trial start date was documented. For the normal renal function participants group, participants were in good health with no clinically significant findings from medical history and physical examination and had eGFR ≥90 mL/min/1.73 m² indicating normal renal function.

Exclusion Criteria: Participants who had significant history or clinical manifestation of any metabolic, allergic, dermatological, hepatic, renal (for normal renal function participants only), hematological, pulmonary, cardiovascular, gastrointestinal, neurological, respiratory, endocrine, or psychiatric disorder, or any other significant disease or disorder, as determined by the investigator (or designee); required dialysis or are expected to require dialysis during the trial period; have a history of drug abuse within the last 2 years; consumed >28 units of alcohol per week for males and >21 units of alcohol for females; consumed more than 5 caffeinated beverages per day; are currently using or have used cannabis within the 3 months prior to starting the trial (recreational or medicinal); consumed >10 nicotine-containing products or equivalent per day; were unwilling to consume/intolerant to ethanol; and had significantly impaired hepatic function at Screening or Check-in on Day −1, defined as any of the following: Serum alanine aminotransferase (ALT) or aspartate aminotransferase (AST)>1.5× upper limit of normal (ULN), Total bilirubin (TBL)>1×ULN, and International normalized ratio (INR)>1.27, were not eligible to enter the trial.

Treatment Period Assessments: Participants who continued to satisfy all inclusion criteria and none of the exclusion criteria, were enrolled to receive the oromucosal spray. The following assessments were performed: physical examination, vital signs, 12-lead ECG, concomitant medications recorded, and AEs were reviewed. Clinical laboratory samples included chemistry, coagulation, hematology, and samples for PK analysis.

Pharmacokinetic (PK) Assessments: From Day 1, blood sampling (total of 14 blood samples) was collected for PK analysis, in which the sampling times included one prior to dosing (t=0) and at the following time points after dosing: 0.25, 0.5, 1, 2, 3, 4, 6, 8, 12, 18, 24, 36, and 48 hours. Urine samples were collected per participant and included prior to dosing for the baseline PK analysis and all urine passed at 0 to 4, 4 to 8, 8 to 12, 12 to 24, and 24 to 48 hours after dosing. PK parameter estimates were evaluated to assess the changes in PK parameters in terms of THC, 11-OH-THC, CBD, and 7-COOH-CBD in steady-state concentrations, following 4 sprays. Logarithmic scale transformed Cₘₐₓ, AUC₍₀₋₀₎, and AUC₍₀₋₁₎ parameters were analyzed using a linear mixed-effects model with a fixed effect for treatment and a random effect for participant. The treatment differences were back transformed to present the ratios of geometric means and the corresponding 90% confidence intervals (CIs). For Tₘₐₓ, nonparametric analysis of the same comparisons were performed using a Wilcoxon signed-rank test. Medians and median differences between the treatments were presented along with the approximate 90% CI for the median difference. Renal Clearance (CLR) and apparent clearance of drug from plasma (CL/F), apparent volume of distribution (Vz/F), and fraction of the systemically available drug excreted into the urine (fe) of the THC, CBD, and/or metabolites of THC and CBD were listed and summarized as appropriate.

Safety Follow-Up Visit Assessments: The following assessments were performed: physical examination, vital signs, 12-lead ECG, adverse events (AEs), and concomitant medications recorded. The investigator or delegate completed the C-SSRS. Clinical laboratory samples included chemistry, hematology, urinalysis, ECG, cortisol, and vital signs.

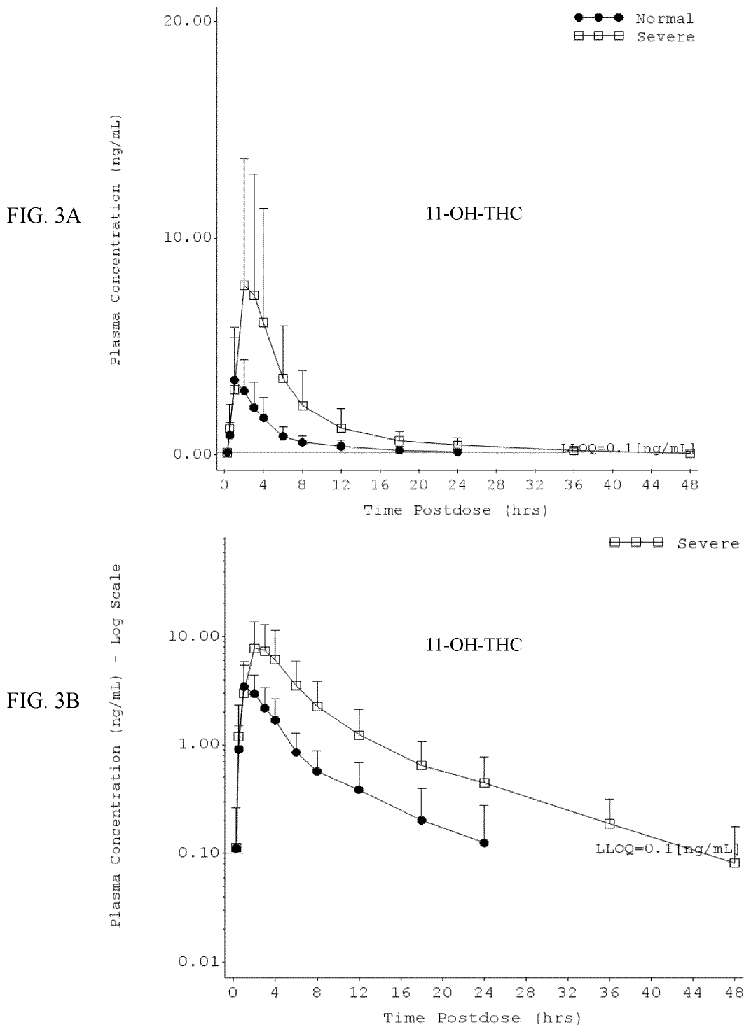

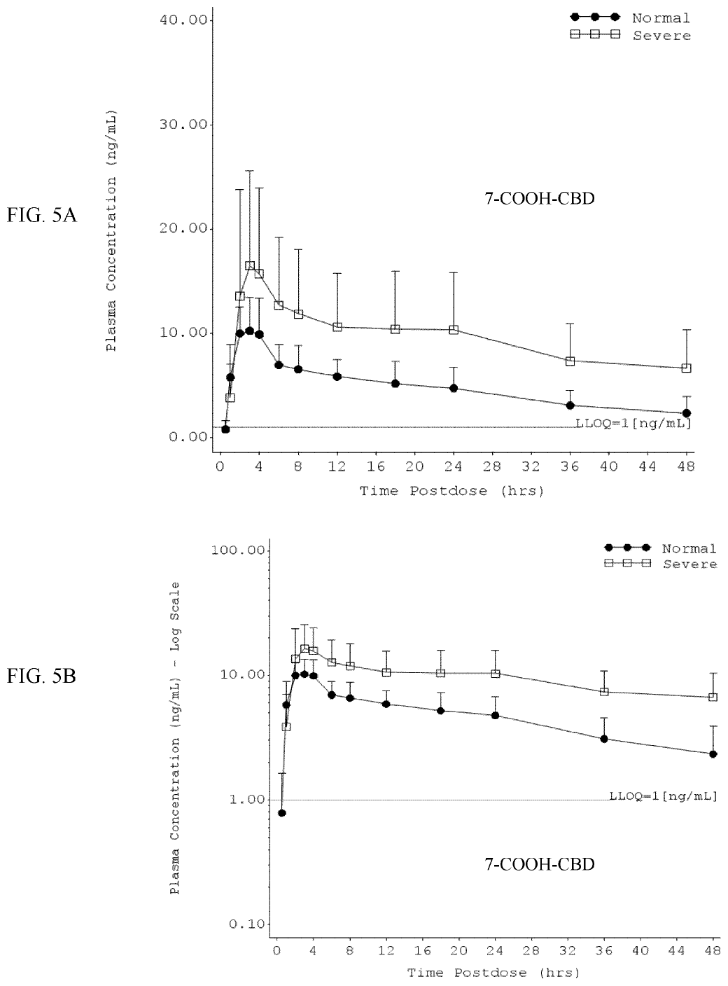

Result: FIGS. 1A-B and 2A-B show the mean plasma concentrations of THC and CBD, respectively, following administration of oromucosal sprays in participants with severe renal impairment (renally impaired) or in participants with normal renal function (normal). As shown in FIGS. 1A-B and 2A-B, plasma levels of THC and CBD over time were elevated in patients with renal impairment. Much higher mean plasma concentrations over time of 11-OH-THC but not 11-COOH-THC, compared to those of THC and CBD, were observed in renally impaired participants (FIGS. 3A-B and 4A-B). Similarly, greater plasma exposure levels of CBD metabolite 7-OH CBD but not 7-COOH CBD were observed in renally impaired compared with the normal renal function participants (FIGS. 5A-B and 6A-B), as apparent from approximately 3-fold increase in AUCₗₐₛₜ of 7-OH-CBD but <2-fold of 7-COOH-CBD. Unexpectedly, the increase in AUC of the OH-metabolites of CBD (7-OH CBD) and THC (11-OH-THC) occurred to a greater extent, compared to the increase in plasma levels of the parent molecules or the COOH-metabolites.

The following data (TABLES A-C) summarize the PK parameters in patients with severe renal impairment and in their matched normal control participants.

TABLE A
Mixed Model of Primary Pharmacokinetic Parameters and Renal Function

| | | Geometric | Geometric LS | | |
|---|---|---|---|---|---|
| | | LS Mean - | Mean - | Geometric LS Mean Ratio | |
| | | Severe Renal | Normal Renal | (Severe RI/Normal) * | |
| | PK Parameter | Impairment | Function | Estimate | 90% CI |
| THC | Cₘₐₓ (ng/mL) | 2.23 | 2.79 | 0.8 | (0.456, 1.41) |
| | AUCₗₐₛₜ | 8.34 | 6.23 | 1.34 | (0.733, 2.45) |
| | (h*ng/ml) | | | | |
| CBD | Cₘₐₓ (ng/mL) | 1.17 | 1.06 | 1.11 | (0.536, 2.28) |
| | AUCₗₐₛₜ | 4.82 | 2.47 | 1.95 | (0.821, 4.65) |
| | (h*ng/mL) | | | | |
| 11-OH-THC | Cₘₐₓ (ng/mL) | 7.28 | 3.42 | 2.13 | (1.33, 3.4) |
| | AUCₗₐₛₜ | 47.9 | 15 | 3.18 | (1.87, 5.42) |
| | (h*ng/mL) | | | | |
| 7-OH-CBD | Cₘₐₓ (ng/mL) | 1.16 | 0.617 | 1.89 | (1.31, 2.73) |
| | AUCₗₐₛₜ | 7.62 | 1.66 | 4.59 | (2.64, 7.97) |
| | (h*ng/mL) | | | | |
| 11-COOH- | Cₘₐₓ (ng/mL) | 28.8 | 25.1 | 1.15 | (0.808, 1.64) |
| THC | AUCₗₐₛₜ | 335 | 265 | 1.26 | (0.893, 1.79) |
| | (h*ng/mL) | | | | |
| 7-COOH- | Cₘₐₓ (ng/mL) | 14.5 | 10.4 | 1.4 | (0.896, 2.18) |
| CBD | AUCₗₐₛₜ | 399 | 208 | 1.92 | (1.24, 2.98) |
| | (h*ng/mL) | | | | |

* Change in AUCₗₐₛₜ between Severe RI and normal is considered significant when the ratio is >=2.0 plus the 90% CI is outside of 0.5-2.0.

TABLE B
Paired T-Test for Comparison of Primary Pharmacokinetic
Parameters between Renal Function Groups

| | | Difference (Ln (Severe RI) − | | |
|---|---|---|---|---|
| | | Ln (Normal)) | | |
| | PK Parameter | Mean | SD | p-value* |
| THC | Cₘₐₓ (ng/mL) | −0.2227 | 0.8395 | 0.4775 |
| | AUCₗₐₛₜ | 0.2928 | 0.6680 | 0.2550 |
| CBD | (h*ng/mL) | | | |
| | Cₘₐₓ (ng/mL) | 0.1014 | 1.1015 | 0.8020 |
| | AUCₗₐₛₜ | 0.6694 | 1.1226 | 0.1355 |
| | (h*ng/mL) | | | |
| 11-OH-THC | Cₘₐₓ (ng/mL) | 0.7560 | 0.7970 | 0.0314 |
| | AUCₗₐₛₜ | 1.1573 | 0.7562 | 0.0034 |
| | (h*ng/mL) | | | |
| 7-OH-CBD | Cₘₐₓ (ng/mL) | 0.6350 | 0.6250 | 0.0239 |
| | AUCₗₐₛₜ | 1.5240 | 0.7025 | 0.0005 |
| | (h*ng/mL) | | | |
| 11-COOH- | Cₘₐₓ (ng/mL) | 0.1399 | 0.6721 | 0.5746 |
| THC | AUCₗₐₛₜ | 0.2347 | 0.6594 | 0.3476 |
| | (h*ng/mL) | | | |
| 7-COOH- | Cₘₐₓ (ng/mL) | 0.3348 | 0.7801 | 0.2642 |
| CBD | AUCₗₐₛₜ | 0.6518 | 0.7887 | 0.0521 |
| | (h*ng/mL) | | | |

*p value <= 0.05 is considered statistically significant change

TABLE C
Summary

| | AUC₀₋ₜₗₐₛₜ Fold Change* from Normal | | | | | |
|---|---|---|---|---|---|---|
| Renal | | | | 11-COOH- | | |
| Impairment | THC | CBD | 11-OH-THC | THC | 7-OH-CBD | 7-COOH-CBD |
| Severe | 1.34 | 1.95 | 3.18 | 1.26 | 4.59 | 1.92 |

As shown above in Table C, oral administration of THC and CBD via oromucosal spray of was associated with a higher exposure rate in the renally impaired patient group compared to the normal control participant group. Notably, the AUC of OH-metabolites of THC and CBD (11-OH-THC and 7-OH-CBD, respectively) increased by 3.18-fold and 4.59-fold higher, respectively. Such magnitude increase in AUC for the OH-metabolites of both THC and CBD is statistically significant with p values <0.05 (Table B), indicating a dose reduction in patients with moderate or severe renal impairment. In contrast, CBD, THC and the COOH metabolites of CBD (7-COOH CBD) or THC (11-COOH THC) had less than 2-fold increase in AUC in the renally impaired group compared to the normal group, which is not considered statistically significant change based on the p values >0.05 (Table B).

The result of this study is unexpected in that the parent molecules (CBD and THC) and their OH-metabolites (7-OH CBD and 11-OH THC) seemed to exhibit differential pharmacokinetic parameters in participants with severe renal impairment. More specifically, and referring to TABLES A-C, the OH-metabolites were associated with higher plasma concentrations and prolonged residence time in the system of the renally impaired group as compared to the parent molecules. If renal impairment had an impact on the overall renal excretion system (or hepatic drug metabolism system), one would expect that both the parent molecules and the metabolites would be affected similarly, but that is not the case here. It is not known why the rates of systemic exposure of the parent compounds or the COOH metabolites versus the OH-metabolites were not equally affected. This differential effect was not observed in previous studies conducted with highly purified CBD (>99% pure CBD) obtained from a botanical source or previous studies in patients with severe hepatic impairment but having normal renal function.

No AEs of special interest (AESI), no serious adverse events (SAE), and no deaths were reported for any of the subjects enrolled in this study. Overall, the means of laboratory parameters, vital signs, and ECGs for the different treatments were comparable. In addition, the mean for all subjects at screening and post-study of evaluated parameters were comparable.

Conclusions: 4 sprays (10.8 mg CBD and 10 mg THC) resulted in greater systemic exposure of CBD, THC, and their metabolites, in the patients with severe renal impairment as compared to the participants with normal renal function. Approximately 1.9-fold increase in AUC for THC, CBD, and the carboxylic acid metabolites, and 3- to 5-fold higher AUC for 7-OH CBD and 11-OH THC were observed in subjects with severe renal impairment compared with subjects with normal renal function. Qualitatively similar increases in exposure may be expected patients with moderate renal impairment, and therefore similar dosing modifications are recommended. Given the slower time to reach its peak concentration, higher peak concentrations of metabolites, and a lower apparent clearance rate in the renally impaired participants, the number of sprays administered to patients with moderate or severe renal impairment is modified as described herein.

As discussed above, the observed AUC fold increase of the parent THC or CBD was less than 2-fold in the patient with severe renal impairment compared to normal participants. Fold changes less than 2 are not considered to be clinically relevant. Accordingly, if the recommended dosage is determined solely based on the results for CBD and THC, no dosage adjustment would be recommended for the renally impaired patients. However, the results above also show an unexpected 3- to 5-fold increase in AUC of active metabolites 7-OH CBD and 11-OH THC, indicating that an adjustment in the daily dosage for the patients with severe and moderate renal impairment is needed. Based on the observed AUC fold change results for THC, CBD, and THC and CBD metabolites, patients with moderate and severe renal impairment would benefit from taking reduced doses (e.g., a maximum of 6 sprays). Such a dose reduction balances the need to achieve clinically relevant plasma concentrations of the parent compounds THC and CBD and pharmacologically active metabolites (efficacy), with the need to avoid elevated plasma levels that present an unacceptable risk to the patients (safety).

### Example 2: Trial to Investigate the Pharmacokinetics in Participants with Mild or Moderate Renal Impairment

Design: This trial follows the same protocol described in Example 1. As described above, participants enter the screening period (Day −28 to −1). During the screening period, participants will be categorized into one of the three groups: 1) participants with normal renal function; 2) participants with mild renal impairment; or 3) participants with moderate renal impairment. The normal control participants having an estimated glomerular filtration rate [eGFR] ≥90 mL/min/1.73 m² are individually matched to participants with mild ([eGFR] between 60 mL/min/1.73 m² and 89 mL/min/1.73 m²) or moderate ([eGFR] between 30 and 59 mL/min/1.73 m²) renal impairment with respect to age (within the decile or ±5 years, whichever is less), sex, and BMI (±10% BMI). From Day −1, screened participants who continue to meet eligibility criteria are admitted to the clinical research facility and placed under observation continually until 48 hours postdose. On Day 1, participants receive 4 sprays, containing a total amount of 10.8 mg THC and 10 mg CBD.

At 48-hour postdose, the following PK parameter estimates will be evaluated to assess the effect of nabiximols dosing regimen on participants with mild or moderate renal impairment: logarithmic scale transformed Cₘₐₓ, AUC₍₀₋₀₎, AUC₍₀₋₁₎, Tₘₐₓ, CLR, CL/F, Vz/F, and fe of the THC, CBD, and/or metabolites of THC and CBD in steady-state concentrations.

### Example 3: Trial to Investigate the Pharmacokinetics of Varying Doses in Participants with Varying Degrees of Renal Impairment

Design: This single-dose phase 1 trial follows the same protocol described in Example 1. The primary objectives of this study are to investigate the effect of administering different numbers of nabiximols oromucosal sprays to the participants with mild, moderate, or severe renal impairment. The participants will be screened and put into one of four groups based on their renal function status: 1) participants with normal renal function (having an estimated glomerular filtration rate [eGFR]≥90 mL/min/1.73 m²); 2) participants with mild renal impairment ([eGFR] between 60 and 89 mL/min/1.73 m²); 3) participants with moderate renal impairment ([eGFR] between 30 and 59 mL/min/1.73 m²); or 4) participants with severe renal impairment ([eGFR]<30 mL/min/1.73 m²). The participants with normal renal function will be individually matched to the participants with renal impairment, and screened participants who continue to meet eligibility criteria on Day −1 are enrolled into the trial. On Day 1, participants receive a single dose of varying number of sprays, as shown in Table H.

TABLE H
Potential Dosing of Nabiximols (in a single setting)

| # of Sprays | Renal Function Status | Total Adm. THC | Total Adm. CBD |
|---|---|---|---|
| 1 | Normal | 2.7 mg | 2.5 mg |
| 1 | Mild renal impairment | 2.7 mg | 2.5 mg |
| 1 | Moderate renal impairment | 2.7 mg | 2.5 mg |
| 1 | Severe renal impairment | 2.7 mg | 2.5 mg |
| 2 | Normal | 5.4 mg | 5.0 mg |
| 2 | Mild renal impairment | 5.4 mg | 5.0 mg |
| 2 | Moderate renal impairment | 5.4 mg | 5.0 mg |
| 2 | Severe renal impairment | 5.4 mg | 5.0 mg |
| 3 | Normal | 8.1 mg | 7.5 mg |
| 3 | Mild renal impairment | 8.1 mg | 7.5 mg |
| 3 | Moderate renal impairment | 8.1 mg | 7.5 mg |
| 3 | Severe renal impairment | 8.1 mg | 7.5 mg |
| 4 | Normal | 10.8 mg | 10.0 mg |
| 4 | Mild renal impairment | 10.8 mg | 10.0 mg |
| 4 | Moderate renal impairment | 10.8 mg | 10.0 mg |
| 4 | Severe renal impairment | 10.8 mg | 10.0 mg |
| 5 | Normal | 13.5 mg | 12.5 mg |
| 5 | Mild renal impairment | 13.5 mg | 12.5 mg |
| 5 | Moderate renal impairment | 13.5 mg | 12.5 mg |
| 5 | Severe renal impairment | 13.5 mg | 12.5 mg |
| 6 | Normal | 16.2 mg | 15.0 mg |
| 6 | Mild renal impairment | 16.2 mg | 15.0 mg |
| 6 | Moderate renal impairment | 16.2 mg | 15.0 mg |
| 6 | Severe renal impairment | 16.2 mg | 15.0 mg |
| 7 | Normal | 18.9 mg | 17.5 mg |
| 7 | Mild renal impairment | 18.9 mg | 17.5 mg |
| 7 | Moderate renal impairment | 18.9 mg | 17.5 mg |
| 7 | Severe renal impairment | 18.9 mg | 17.5 mg |
| 8 | Normal | 21.6 mg | 20.0 mg |
| 8 | Mild renal impairment | 21.6 mg | 20.0 mg |
| 8 | Moderate renal impairment | 21.6 mg | 20.0 mg |
| 8 | Severe renal impairment | 21.6 mg | 20.0 mg |
| 9 | Normal | 24.3 mg | 22.5 mg |
| 9 | Mild renal impairment | 24.3 mg | 22.5 mg |
| 9 | Moderate renal impairment | 24.3 mg | 22.5 mg |
| 9 | Severe renal impairment | 24.3 mg | 22.5 mg |
| 10 | Normal | 27.0 mg | 25.0 mg |
| 10 | Mild renal impairment | 27.0 mg | 25.0 mg |
| 10 | Moderate renal impairment | 27.0 mg | 25.0 mg |
| 10 | Severe renal impairment | 27.0 mg | 25.0 mg |
| 11 | Normal | 29.7 mg | 27.5 mg |
| 11 | Mild renal impairment | 29.7 mg | 27.5 mg |
| 11 | Moderate renal impairment | 29.7 mg | 27.5 mg |
| 11 | Severe renal impairment | 29.7 mg | 27.5 mg |
| 12 | Normal | 32.4 mg | 30.0 mg |
| 12 | Mild renal impairment | 32.4 mg | 30.0 mg |
| 12 | Moderate renal impairment | 32.4 mg | 30.0 mg |
| 12 | Severe renal impairment | 32.4 mg | 30.0 mg |

Total amount of THC or CBD in a single dose

Pharmacokinetic parameter estimates are evaluated to assess the change in PK parameters of CBD, THC, 7-OH-THC, 7-OH-CBD, 11-COOH-THC, and 7-COOH-CBD, following a single dose of nabiximols.

Log transformed Cₘₐₓ, AUC₍₀₋₀₎, and AUC₍₀₋ₜ₎ parameters for nabiximols are analyzed using a linear mixed-effects model with a fixed effect for treatment and a random effect for participant. The treatment differences are back transformed to present the ratios of geometric means and the corresponding 90% confidence intervals (CIs).

For Tₘₐₓ, nonparametric analysis of the same comparisons is performed using a Wilcoxon signed-rank test. Medians and median differences between the treatments are presented along with the approximate 90% CI for the median difference.

Geometric mean ratios and 90% CIs are used to estimate the magnitude of any interaction and will be interpreted based on clinical relevance.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

What is claimed is:

1. A method of treating spasticity in a patient with multiple sclerosis, and the patient has moderate or severe renal impairment, comprising orally administering to the patient a maximum of 6 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

2. A method of treating neuropathic pain in a patient with multiple sclerosis, and the patient has moderate or severe renal impairment, comprising orally administering to the patient a maximum of 6 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

3. A method of treating pain in a patient with advanced cancer, and the patient has moderate or severe renal impairment, comprising orally administering to the patient a maximum of 6 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

4. The method of claims any one of claims 1-3, wherein the patient has severe renal impairment.

5. The method of claim 4, wherein the patient with severe renal impairment is administered 1 spray per day.

6. The method of claim 4, wherein the patient with severe renal impairment is administered 2 spray per day.

7. The method of claim 4, wherein the patient with severe renal impairment is administered 3 spray per day.

8. The method of claim 4, wherein the patient with severe renal impairment is administered 4 spray per day.

9. The method of claim 4, wherein the patient with severe renal impairment is administered 5 spray per day.

10. The method of claim 4, wherein the patient with severe renal impairment is administered 6 spray per day.

11. The method of claims any one of claims 1-3, wherein the patient has moderate renal impairment.

12. The method of claim 11, wherein the patient with moderate renal impairment is administered 1 spray per day.

13. The method of claim 11, wherein the patient with moderate renal impairment is administered 2 spray per day.

14. The method of claim 11, wherein the patient with moderate renal impairment is administered 3 spray per day.

15. The method of claim 11, wherein the patient with moderate renal impairment is administered 4 spray per day.

16. The method of claim 11, wherein the patient with moderate renal impairment is administered 5 spray per day.

17. The method of claim 11, wherein the patient with moderate renal impairment is administered 6 spray per day.

18. A method of treating spasticity in patient with multiple sclerosis, and the patient has mild renal impairment, comprising orally administering to the patient a maximum of 12 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

19. A method of treating neuropathic pain in a patient with multiple sclerosis, and the patient has mild renal impairment, comprising orally administering to the patient a maximum of 12 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

20. A method of treating pain in a patient with advanced cancer, and the patient has mild renal impairment, comprising orally administering to the patient a maximum of 12 sprays per day of an oromucosal spray comprising Δ-9-tetrahydrocannabinol (THC) and cannabidiol (CBD), wherein the THC and CBD are present at a weight ratio ranging from 0.5:1.5 to 1.5:0.5.

21. The method of any one of claims 18-20, wherein the patient with mild renal impairment is administered 1 spray per day.

22. The method of any one of claims 18-20, wherein the patient with mild renal impairment is administered 2 sprays per day.

23. The method of any one of claims 18-20, wherein the patient with mild renal impairment is administered 3 sprays per day.

24. The method of any one of claims 18-20, wherein the patient with mild renal impairment is administered 4 sprays per day.

25. The method of any one of claims 18-20, wherein the patient with mild renal impairment is administered 5 sprays per day.

26. The method of any one of claims 18-20, wherein the patient with mild renal impairment is administered 6 sprays per day.

27. The method of any one of claims 18-20, wherein the patient with mild renal impairment is administered 7 sprays per day.

28. The method of any one of claims 18-20, wherein the patient with mild renal impairment is administered 8 sprays per day.

29. The method of any one of claims 18-20, wherein the patient with mild renal impairment is administered 9 sprays per day.

30. The method of any one of claims 18-20, wherein the patient with mild renal impairment is administered 10 sprays per day.

31. The method of any one of claims 18-20, wherein the patient with mild renal impairment is administered 11 sprays per day.

32. The method of any one of claims 18-20, wherein the patient with mild renal impairment is administered 12 sprays per day.

33. The method of any one of the preceding claims, wherein the oromucosal spray delivers about 50 μL to 200 μL per spray.

34. The method of any one of the preceding claims, wherein the oromucosal spray delivers about 100 μL per spray.

35. The method of any one of the preceding claims, wherein the patient is administered about 2 to about 3 mg of THC and about 2 to about 3 mg of CBD per spray.

36. The method of any one of the preceding claims, wherein the patient is administered about 2.25 to about 2.75 mg of THC and about 2.25 to about 2.75 mg of CBD per spray.

37. The method of any one of the preceding claims, wherein the patient is administered about 2.7 mg THC and about 2.5 mg CBD per spray.

38. The method of any one of the preceding claims, wherein the oromucosal spray comprises from 20-30 mg/mL THC and from 20-30 mg/mL CBD.

39. The method of any one of the preceding claims, wherein the oromucosal spray comprises 22.5 to 27.5 mg/mL THC and from 22.5 to 27.5 mg/mL CBD.

40. The method of any one of the preceding claims, wherein the oromucosal comprises about 27 mg/mL THC and about 25 mg/mL CBD.

41. The method of any one of the preceding claims, wherein the THC and CBD are present in a weight ratio of from about 0.9:1.1 to about 1.1:0.9.

42. The method of any one of the preceding claims, wherein the THC and CBD are present in a weight ratio of about 1.1:0.9.

43. The method of any one of the preceding claims, wherein the oromucosal spray comprises ethanol.

44. The method of any one of the preceding claims, wherein the oromucosal spray comprises propylene glycol.

45. The method of any one of the preceding claims, wherein the oromucosal spray comprises peppermint oil.

46. A method of managing spasticity in a renally impaired patient with multiple sclerosis, neuropathic pain in a renally impaired patient with multiple sclerosis, or pain in a renally impaired patient with advanced cancer, comprising:
(a) determining the patient's renal impairment status;
(b) (i) if the patient has moderate or severe renal impairment, administering a maximum of 6 sprays per day of an oromucosal comprising Δ-9-tetrahydrocannabinol (THC) and cannabidiol (CBD); or
(ii) if the patient has mild renal impairment, administering a maximum of 12 sprays per day of an oromucosal comprising Δ-9-tetrahydrocannabinol (THC) and cannabidiol (CBD),
wherein the THC and CBD are present at a weight ratio ranging from 0.5:1.5 to 1.5:0.5.

47. The method of claim 46, wherein the patient with mild renal impairment is administered about 2.5 mg to about 32.4 mg of THC and about 2.5 mg to about 32.4 mg of CBD per day.

48. The method of claim 46, wherein the patient with moderate renal impairment is administered about 2.5 mg to about 16.2 mg of THC and about 2.5 mg to about 16.2 mg of CBD per day.

49. The method of claim 46, wherein the patient with severe renal impairment is administered about 2.5 mg to about 16.2 mg of THC and about 2.5 mg to about 16.2 mg of CBD per day.

50. The method of any one of claim 1, 3-18, or 21-49, wherein the patient has moderate to severe spasticity.

51. The method of any one of claim 1, 4-18, or 21-50, wherein the patient is administered the oromucosal spray in combination with one or more anti-spasticity medications.

52. The method of claim 51, wherein the one or more anti-spasticity medications is baclofen, tizanidine, dantrolene sodium, diazepam, clonazepam, and gabapentin.

53. The method of any one of claim 3-18 or 20-52, wherein the patient with advanced cancer has moderate to severe pain.

54. The method of claim 53, wherein patient experiences moderate to severe pain during the highest tolerated of a strong opioid therapy.

55. The method of claim 54, wherein the strong opioid is tramadol, buprenorphine, methadone, diamorphine, fentanyl, hydromorphone, morphine, oxycodone, or pethidine.

56. The method of any one of the preceding claims, wherein the patient is an adult.

57. The method of claim 56, wherein the patient is 18-65 years old.

58. The method of anyone of the preceding claims, wherein the oromucosal spray comprises a botanical drug substance comprising THC and CBD.

59. The method of any one of the preceding claims, wherein the oromucosal spray further comprises one or more cannabinoids in addition to THC and CBD.

60. The method of claim 59, wherein the one or more cannabinoids are cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof.

61. The method of claim any one of the preceding claims, wherein the oromucosal spray further comprises one or more terpenes.

62. The method of any one of the preceding claims, wherein the oromucosal spray further comprises one or more sesquiterpenes.

63. The method of claim 61 or 62, wherein the one or more terpenes or sesquiterpenes are beta-farnesene, selina-3,7(11)-diene, guaia-3,9-diene, trans-caryophyllene, alpha-caryophyllene, trans-nerolidol, myrcene, trans-phytol, squalene, alpha-tocopherol, or a combination thereof.

64. The method of claim of any one of the preceding claims, wherein the oromucosal spray further comprise one or more sterols.

65. The method of claim 64, wherein the one or more sterols are beta-sitosterol, beta-amyrin, campesterol, lupeol, or combinations thereof.

66. The method of claim 46, wherein the patient with mild renal impairment is administered 1, 2, 3, 4, 5 or 6 sprays per day of the oromucosal spray.

67. The method of claim 46, wherein the patient with moderate renal impairment is administered 1, 2, 3, 4, 5 or 6 sprays per day of the oromucosal spray.

68. The method of claim 46, wherein the patient with severe renal impairment is administered 1, 2, 3, 4, 5, 6, 7, 8, 9, 10, 11, or 12 sprays per day of the oromucosal spray.

69. The method of any one of claims 1-10, 33-46, and 49-68, wherein the patient with several renal impairment has end stage renal disease (ESRD).

70. The method of claim 69, wherein the patient with ESRD does not receive dialysis.
